(19) United States
(12) Patent Application Publication
(10) Pub. No.: US 2012/0108592 A1
(43) Pub. Date: May 3, 2012

(54) PHARMACEUTICAL COMPOSITIONS AND METHODS OF USE OF SALICYLANILIDES FOR TREATMENT OF HEPATITIS VIRUSES

(75) Inventors: J. Edward Semple, Tampa, FL (US); Jean-Francois Rossignol, St. Petersburg, FL (US)

(73) Assignee: Romark Laboratories, L.C.

(21) Appl. No.: 13/284,232

(22) Filed: Oct. 28, 2011

Related U.S. Application Data
(60) Provisional application No. 61/408,215, filed on Oct. 29, 2010.

(51) Int. Cl. A61K 31/5375; A61K 31/196; A61K 31/365; A61K 31/366; A61K 31/222; A61K 31/24; A61K 31/404; A61K 31/37; A61K 31/4025; A61K 31/4965; A61K 31/50; A61K 31/4164; A61K 31/4409; A61K 31/4406; A61K 31/4458; A61K 31/4462; A61K 31/4465; A61K 31/495; A61P 31/14; A61P 31/20; A61K 31/245; A61K 31/265; A61K 31/4245; A61K 31/41; A61K 31/167

(52) U.S. Cl. 514/237.8; 514/622; 514/563; 514/465; 514/452; 514/546; 514/534; 514/415; 514/457; 514/422; 514/255.06; 514/247; 514/400; 514/357; 514/330; 514/255.01; 514/327; 514/535; 514/512; 514/364; 514/490; 514/381

(57) ABSTRACT

A new class of salicylanilides is described. These compounds show strong activity against hepatitis viruses.

### CROSS-REFERENCE TO RELATED APPLICATIONS

This application claims priority from U.S. Provisional Application No. 61/408,215, filed Oct. 29, 2010.

### STATEMENT REGARDING FEDERALLY SPONSORED RESEARCH OR DEVELOPMENT

The invention disclosed herein was made with Government support under NIAID contract NO1-AI-30046 to Georgetown University Medical Center. Accordingly, the U.S. Government may have certain rights in this invention.

### FIELD OF THE INVENTION

The present invention is directed to new salicylanilide compounds and pharmaceutical compositions thereof and their methods of use for the treatment of disease. Methods of inhibition of viral pathogen activity in a human or animal subject are also provided for the treatment of diseases, such as hepatitis C virus (HCV), hepatitis B virus (HBV) and related viral pathogens.

### BACKGROUND

Hepatitis B Virus (HBV) and Hepatitis C Virus (HCV) are major public health problems, causing more than an estimated 500 million chronic infections worldwide. Both viruses are a source of significant progressive liver disease, and are the major risk factors for nearly all cases of primary hepatocellular carcinoma [Chen, S. L., Morgan, T. R., Int. J. Med. Sci. 3, 47-52 (2006); Lavanchy, D., J. Viral. Hepat. 11, 97-107 (2004); Wong, S. N., Lok, A. S., Curr. Opin. Gastroenterol. 22, 241-247 (2006)]. Licensed standards of care for both viral infections, while effective in many cases, are sub-optimal and do not result in virologic or clinical ‘cures’ in most individuals (see Wong et al., (2006)). The development of drug-resistance in HBV, including strains carrying resistance to multiple licensed agents is an emerging clinical problem, and drug-resistance for future HCV therapies is predicted to be a significant clinical issue [Tomei, L. et al., Antivir. Chem. Chemother. 16, 225-245 (2005); Tong et al., Antivir. Res. 70, 28-38 (2006); Yim et al., Hepatology 44, 703-712 (2006)].

Thiazolide compounds such as nitazoxanide (NTZ) are anti-infective and possess activity against anaerobic bacteria, protozoa and viruses [Rossignol, J. F. Santoro, M. G. et al., J. Biol. Chem., 284, 29798-29808 (2009); Korba, B. E. et al. Antiviral Res. 77, 56-63 (2008); Fox, L. M., Saravolatz, L. D., Clin. Infect. Dis. 40, 1173-1180 (2005); Pankuch, G. A., Appelbaum, P. C., Antimicrob. Agents Chemother. 50, 112-117 (2006); Rossignol, J. F. et al., Lancet 368, 124-129 (2006); Rossignol and El-Gohary, Aliment. Pharmacol. Ther. 24, 1423-1430 (2006)]. Originally developed as a treatment of intestinal protozoan infections, the antiviral properties of NTZ were discovered during the course of its development for treating cryptosporidiosis in patients with acquired immune deficiency syndrome (AIDS).

NTZ is marketed in the United States for treating diarrhea and enteritis caused by Cryptosporidium spp or Giardia lamblia in adults and children down to 12 months of age (Alinia®, Romark Laboratories, Tampa, Fla. USA). Clinical trials have demonstrated effectiveness of NTZ in treating diarrhea and enteritis associated with enteric protozoan infections caused by Cryptosporidium spp, G. lamblia, Entamoeba histolytica and Blastocystis hominis [Amadi et al., Lancet 360, 1375-1380. (2002); Oritz et al., Aliment. Pharmacol. Ther. 15, 1409-1415. (2001); Rossignol. J. F. et al., J. Infect. Dis. 184, 381-384 (2001); Clin. Gastroenterol. Hepatol. 3, 987-991. (2005), Clin. Gastroenterol. Hepatol. 4, 320-324. (2006)]. Recent randomized double-blind clinical trials have demonstrated effectiveness of NTZ in treating Clostridium difficile colitis in adults, rotavirus gastroenteritis in young children, and rotavirus and norovirus gastroenteritis in adults [Musher et al., Clin. Infect. Dis. 43, 421-427 (2006); Rossignol et al, Lancet 368, 124-129 (2006); Rossignol and El Gohary, Aliment. Pharmacol. Ther. 24, 1423-1430 (2006)].

The mechanism of action of NTZ against anaerobic organisms is attributed to interference with pyruvate:ferredoxin oxidoreductase (PFOR) enzyme-dependent electron transfer reactions, which are essential for anaerobic energy metabolism [Hoffman et al., Antimicrob. Agents Chemother. 51, 868-876 (2006)]. While the mechanism of antiviral activity of NTZ against hepatitis B and C has not been fully elucidated, recent studies have suggested that NTZ may play a role in the modulation of protein in a host cell. For RNA viruses, such as influenza A [Rossignol J. F. and Santoro M. G. (2009)] and HCV [Elazar M., et al. Gastroenterology, 137, 1827-1835 (2009)], NTZ selectively inhibits viral glycoproteins at the post-translational level, preventing final assembly of the virus before its exit out of the cell to infect another cell. Also in HCV, NTZ and other thiazolide analogs cause an increase in eIF2α phosphorylation induced by PKR activation, which are key mediators of intracellular host antiviral activity [Elazar M. (2009].

Following oral administration of a 500 mg tablet, NTZ is partially absorbed from the gastrointestinal tract and rapidly hydrolyzed in plasma to form its active circulating metabolite, tizoxanide (TIZ). NTZ is not detected in plasma. Maximum serum concentrations of TIZ, reach approximately 10 μg/mL (37 μM) [Stockis et al., Int. J. Clin. Pharmacol. Ther. 40, 221-227 (2002)] following oral administration of one 500 mg NTZ tablet (Alinia®) with food. TIZ is glucurono-conjugated in the liver and excreted in urine and bile. Approximately two-thirds of an oral dose pass through the intestinal tract and is excreted in feces as TIZ [Broekhuysen, J. et al., Int. J. Clin. Pharmacol. Ther. 38, 387-394 (2000)]. The elimination half-life of TIZ from plasma is approximately 1.5 hours. TIZ does not inhibit cytochrome P450 enzymes, and therefore, no drug-drug interactions are expected [Broekhuysen et al., 2000; Stockis et al. (2002)]. The most commonly reported side-effects in clinical trials include mild abdominal pain, headache, diarrhea and nausea, which occur at rates similar to those reported for patients receiving placebo. While most of the clinical experience with NTZ has involved a 3 to 14 days treatment cycle, continued use of the drug for periods as long as 4 years have been evaluated in patients with AIDS-related cryptosporidiosis. These studies indicate no significant drug-related adverse events [Fox, L. M., Saravolatz, L. D. (2005); Rossignol and El-Gohary (2006)].

The present application relates generally to the field of salicylanilide compounds. In particular, the hepatitis B and C viral inhibition activity of the inventive salicylanilide compounds having an electron-withdrawing groups in the phenyl ring is compared to the antiviral activity of strucuturally related thiazolides Nitazoxanide and Tizoxanide whose structures are sshown below:

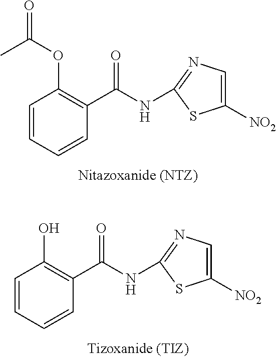

### SUMMARY

The present inventors have found that salicylanilides that comport with Formula I are candidate therapeutics for the treatment of viral infection, for example, infections caused by the hepatitis B and hepatitis C viruses.

The inventive compounds are regarded as structural analogs or isosteres of the thiazolide class of compounds. Described below are methods for synthesizing the inventive compounds, their pharmaceutical compositions and methods for using compositions of the inventive compounds in the treatment of viral pathogens. Also described are methods for using the inventive compounds to inhibit virus in a patient by administering a pharmaceutical composition of said compound.

The present invention discloses methods of use and pharmaceutical compositions of a class of compounds and their pharmaceutically acceptable salts for treating disorders and conditions caused by viral pathogens. The inventive compounds conform to Formula I:

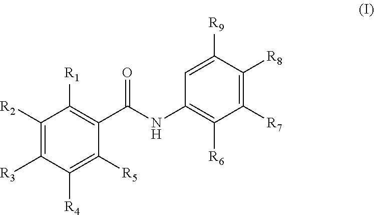

In Formula I, R₁, R₂ or R₃ are each independently selected from the group consisting of hydrogen, duterium, hydroxy, F, Cl, Br, (C₁-C₆)-alkyl, (C₃-C₈)-cycloalkyl, (C₁-C₆)-alkoxy, carboxy, (C₁-C₆)-alkoxycarbonyl, amino, (C₁-C₆)-acylamino, (C₁-C₆)-alkylsulfonylamino, and Q—C(═O)O—, any of which may be optionally substituted.

Alternatively, any two contiguous R₁, R₂ or R₃ moieties together with the atoms to which they are attached form an optionally substituted 5-to 8-membered heterocycloalkyl ring.

Substituents R₄ and R₅ are independently selected from the group consisting of hydrogen, duterium, F, Cl, Br, (C₁-C₆)-alkyl, (C₃-C₈)-cycloalkyl, (C₁-C₆)-alkoxy, (C₂-C₆)-alkenyl, (C₂-C₆)-alkynyl and (C₁-C₁₂)-alkoxycarbonyl, any of which may be optionally substituted.

R₆ through R₉ are independently selected from the group consisting of hydrogen, CN, NO₂, F, Cl, Br, I, (C₁-C₆)-alkyl, (C₂-C₆)-alkenyl, (C₂-C₆)-alkynyl, (C₃-C₈)-cycloalkyl, (C₃-C₈)-cycloalkenyl, (C₁-C₆)-alkoxy, (C₂-C₆)-alkenyloxy, (C₂-C₆)-alkynyloxy, (C₁-C₆)-alkoxyalkyl, (C₃-C₈)-cycloalkoxy, (C₅-C₈)-cycloalkenyloxy, (C₁-C₆)-alkoxyalkylamino, (C₁-C₆)-acyl, (C₁-C₆)-alkylamino, (C₁-C₆)-dialkylamino, carboxy, (C₁-C₆)-alkoxycarbonyl, amido, (C₁-C₆)-alkylamido, (C₁-C₆)-dialkylamido, (C₁-C₆)-haloalkyl, (C₁-C₆)-perhaloalkyl, (C₁-C₆)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₂-C₆)-alkenylsulfonyl, (C₂-C₆)-alkynylsulfonyl, (C₃-C₈)-cycloalkylsulfonyl, (C₃-C₈)-cycloalkylsulfinyl, arylsulfonyl, arylsulfinyl, arylalkylsulfonyl, arylalkylsulfinyl, arylalkenylsulfonyl, heteroarylsulfonyl, heteroarylsulfinyl, heteroarylalkylsulfonyl, heteroarylalkylsulfinyl, heteroarylalkenylsulfonyl, (C₁-C₆)-alkylsulfonamido, N,N′-(C₁-C₆)-dialkylsulfonamido, sulfonamidoalkyl, sulfonamidoaryl, sulfonamidoarylalkyl, sulfonamidoarylalkenyl, aryl, arylalkyl, aryloxy, arylalkoxy, arylthio, arylalkylthio, arylamino, arylalkylamino, arylalkenyl, arylalkynyl, heteroaryl, heteroarylalkyl, heteroarylalkenyl, heteroarylalkynyl, heteroaryloxy, heteroarylalkoxy, heteroarylamino, heteroarylalkylamino, heteroarylthio, heteroarylalkylthio, heteroarylalkylamino, heterocycloalkyl, heterocycloalkenyl, heterocycloalkoxy, and heterocycloalkenyloxy, any of which may be optionally substituted.

Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₁₂)-alkenyl, (C₂-C₁₂)-alkynyl, (C₃-C₈)-cycloalkyl, aryl, arylalkyl, arylalkenyl, heterocycloalkyl, heteroaryl, heteroarylalkyl, and heteroarylalkenyl, any of which may be optionally substituted; or R₁₀ and R₁₁, together with the atoms to which they are attached, may be joined to form an optionally substituted 5-to 8-membered heterocycloalkyl ring; any of which may be optionally substituted.

Compounds according to the present invention possess useful virus inhibiting or modulating activity, and may be used in the treatment or prophylaxis of a disease or condition in which a viral pathogen plays an active role. Thus, in a broad aspect, the present invention also provides pharmaceutical compositions comprising one or more compounds of the present invention together with a pharmaceutically acceptable carrier (e.g., a diluent or excipient), as well as methods of making and using the compounds and compositions. The pharmaceutical composition may comprise an effective amount of the compound for treating HCV, HBV, and other viral infections.

In certain embodiments, the present invention provides methods for inhibiting or modulating a viral pathogen. In other embodiments, the present invention provides methods for treating a viral-mediated disorder in a patient in need of such treatment comprising administering to said patient a therapeutically effective amount of a compound or composition according to the present invention. Also disclosed are methods for treating HCV, HBV and other viral infections comprising administering the disclosed pharmaceutical compositions to a patient in need thereof. For example, the patient may have a chronic HCV infection. The present invention also contemplates the use of compounds disclosed herein for use in the manufacture of a medicament for the treatment of a disease or condition ameliorated by the inhibition or modulation of viral activity.

### DETAILED DESCRIPTION OF THE PREFERRED EMBODIMENTS

Unless otherwise specified, “a” or “an” means “one or more.”

In certain embodiments, methods of use and pharmaceutical compositions of the class of compounds of the present invention have structural Formula I:

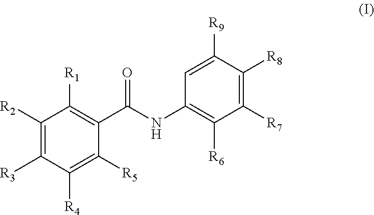

R₁, R₂ or R₃ are each independently selected from the group consisting of hydrogen, duterium, hydroxy, F, Cl, Br, (C₁-C₃)-alkyl, (C₃-C₆)-cycloalkyl, (C₁-C₄)-alkoxy, carboxy, (C₁-C₃)-alkoxycarbonyl, (C₁-C₆)-acylamino, (C₁-C₆)-alkylsulfonylamino, and Q—C(═O)O—, any of which may be optionally substituted. Alternatively, any two contiguous R₁, R₂ or R₃ moieties together with the atoms to which they are attached and joined to form an optionally substituted 5-to 6-membered heterocycloalkyl ring.

R₄ and R₅ are independently selected from the group consisting of hydrogen, duterium, F, Cl, Br, (C₁-C₃)-alkyl, (C₃-C₆)-cycloalkyl, (C₁-C₃)-alkoxy, (C₂-C₄)-alkenyl, and (C₂-C₄)-alkynyl, any of which may be optionally substituted.

R₆ through R₉ are independently selected from the group consisting of hydrogen, CN, NO₂, F, Cl, Br, I, (C₁-C₆)-alkyl, (C₃-C₆)-cycloalkyl, (C₁-C₆)-alkoxy, (C₃-C₆)-cycloalkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, arylsulfonyl, arylsulfinyl, arylalkylsulfonyl, arylalkylsulfinyl, heteroarylsulfonyl, heteroarylsulfinyl, heteroarylalkylsulfonyl, heteroarylalkylsulfinyl, (C₁-C₆)-alkylsulfonamido, N,N′—(C₁-C₆)-dialkylsulfonamido, aryl, arylalkyl, aryloxy, arylalkoxy, arylthio, arylalkylthio, heteroaryl, heteroarylalkyl, heteroaryloxy, heteroarylalkoxy, heteroarylthio, heteroarylalkylthio, heterocycloalkyl, and heterocycloalkoxy, any of which may be optionally substituted.

Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and Ru, together with the atoms to which they are attached, may be joined to form an optionally substituted 5- to 6-membered heterocycloalkyl ring; any of which may be optionally substituted.

In other embodiments, methods of use and pharmaceutical compositions of the class of compounds of the present invention have structural Formula I wherein R1 is selected from the group consisting of hydroxy, carboxy, (C₁-C₃)-alkoxycarbonyl, and Q—C(═O)O—, any of which may be optionally substituted.

R₂ and R₃ are independently selected from the group consisting of hydrogen, hydroxy, duterium, F, Cl, Br, (C₁-C₃)-alkyl, (C₃-C₆)-cycloalkyl, (C₁-C₄)-alkoxy, and Q—C(═O)O—, any of which may be optionally substituted; or R₁ and R₂ may be combined together with the atoms to which they are attached and joined to form an optionally substituted 5-to 6-membered heterocycloalkyl ring.

R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted.

R₆ through R₉ are independently selected from the group consisting of hydrogen, CN, NO₂, F, Cl, Br, I, (C₁-C₆)-alkyl, (C₃-C₆)-cycloalkyl, (C₁-C₆)-alkoxy, (C₃-C₆)-cycloalkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, arylsulfonyl, arylsulfinyl, arylalkylsulfonyl, arylalkylsulfinyl, heteroarylsulfonyl, heteroarylsulfinyl, heteroarylalkylsulfonyl, heteroarylalkylsulfinyl, (C₁-C₆)-alkylsulfonamido, N,N′-(C₁-C₆)-dialkylsulfonamido, aryl, arylalkyl, aryloxy, arylalkoxy, arylthio, arylalkylthio, heteroaryl, heteroarylalkyl, heteroaryloxy, heteroarylalkoxy, heteroarylthio, heteroarylalkylthio, heterocycloalkyl, and heterocycloalkoxy, any of which may be optionally substituted.

Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and R₁₁, together with the atoms to which they are attached, may be joined to form an optionally substituted 5- to 6-membered heterocycloalkyl ring; any of which may be optionally substituted.

In other embodiments, methods of use and pharmaceutical compositions of the class of compounds of the present invention have structural Formula I wherein R₁ is selected from the group consisting of hydroxy and Q—C(═O)O—, which may be optionally substituted.

R₂ and R₃ are independently selected from the group consisting of hydrogen, duterium, F, Cl, (C₁-C₃)-alkyl, and (C₁-C₄)-alkoxy, any of which may be optionally substituted.

R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted.

R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and Ru, together with the atoms to which they are attached, may be joined to form an optionally substituted 5- to 6-membered heterocycloalkyl ring; any of which may be optionally substituted.

In other embodiments, methods of use and pharmaceutical compositions of the class of compounds of the present invention have structural Formula I wherein R₁ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

R₂ and R₃ are independently selected from the group consisting of hydrogen, duterium, F, Cl, (C₁-C₃)-alkyl, and (C₁-C₄)-alkoxy, any of which may be optionally substituted.

R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted and R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

In other embodiments, methods of use and pharmaceutical compositions of the class of compounds of the present invention have structural Formula I R1 is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N1,N2-dimethyl-1,2-ethanediamin-1-yl, N1,N2-diethyl-1,2-ethanediamin-1-yl, N1,N2-dimethyl-1,3-propanediamin-1-yl, and N1-methyl-N2-(2-morpholinoethyl)-1,2-ethanediamin-1-yl.

R2 and R3 are independently selected from the group consisting of hydrogen, F, Cl, methyl, ethyl, isobutyl, methoxy, ethoxy, and isopropoxy, any of which may be optionally substituted.

Substituents R4 and R5 are each independently selected from the group consisting of hydrogen, F, Cl, Br, methyl, isopropyl, cyclopropyl and methoxy, any of which may be optionally substituted and R₆ through R₉ are independently selected from the group consisting of hydrogen, CN, NO₂, F, Cl, Br, I, methyl, methoxy, methoxycarbonyl, butoxycarbonyl, amido, ethylamido, N,N-dimethylamido, trifluoromethyl, tetrafluoroethoxy, methylthio, ethylthiomethyl, methylsulfonyl, t-butylsulfinyl, 2-methylsulfonyl-1-ethyl, 2-methylsulfinylmethyl, cyclopropylsulfonyl, cyclopentylsulfinyl, N-propylsulfonamido, and N,N′-diethylsulfonamido, any of which may be optionally substituted.

In certain embodiments, methods of use and pharmaceutical compositions of the class of compounds of the present invention have structural Formula I wherein R₁ is selected from the group consisting of hydroxy and Q, where R₁₀ is methyl and R₂ and R₄ are each independently selected from the group consisting of hydrogen, F, Cl, methyl, and methoxy.

Substituents R₃ and R₅ are hydrogen and R₆ is selected from the group consisting of hydrogen, F, Cl, Br, methyl, and methylsulfonyl. Additionally, R₇ is selected from the group consisting of hydrogen, F, Cl, Br, NO₂, methyl, trifluoromethyl, methylsulfonyl and methoxycarbonyl.

R₈ is selected from the group consisting of hydrogen, F, NO₂, Cl, I, methylsulfonyl, methoxycarbonyl, tetrafluoroethoxy, and trifluoromethyl and R₉ is selected from the group consisting of hydrogen, Cl, methyl, trifluoromethyl, methoxycarbonyl, and NO₂.

In other embodiments, methods of use and pharmaceutical compositions of the class of compounds of the present invention have structural Formula I wherein R₁ is selected from the group consisting of hydroxy and Q, where R₁₀ is methyl. R₂ through R₅ are hydrogen and R₆ is selected from the group consisting of hydrogen, F, Cl, Br, methyl, and methylsulfonyl.

Substituent R₇ is selected from the group consisting of hydrogen, F, Cl, Br, NO₂, methyl, trifluoromethyl, methylsulfonyl and methoxycarbonyl.

R₈ is selected from the group consisting of hydrogen, F, NO₂, Cl, I, methylsulfonyl, methoxycarbonyl, tetrafluoroethoxy, and trifluoromethyl and R₉ is selected from the group consisting of hydrogen, Cl, methyl, trifluoromethyl, methoxycarbonyl, and NO₂.

In other embodiments, methods of use and pharmaceutical compositions of the class of compounds of the present invention have structural Formula I. R₂ is selected from the group consisting of hydroxy, carboxy, (C₁-C₃)-alkoxycarbonyl, and Q—C(═O)O—, any of which may be optionally substituted.

R₁ and R₃ are each independently selected from the group consisting of hydrogen, hydroxy, duterium, F, Cl, Br, (C₁-C₃)-alkyl, (C₃-C₆)-cycloalkyl, (C₁-C₄)-alkoxy, and Q—C(═O)O—, any of which may be optionally substituted; or R₂ and R₃ may be combined together with the atoms to which they are attached and joined to form an optionally substituted 5-to 6-membered heterocycloalkyl ring.

Substituent groups R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted and R₆ through R₉ are independently selected from the group consisting of hydrogen, CN, NO₂, F, Cl, Br, I, (C₁-C₆)-alkyl, (C₃-C₆)-cycloalkyl, (C₁-C₆)-alkoxy, (C₃-C₆)-cycloalkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthio alkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, arylsulfonyl, arylsulfinyl, arylalkylsulfonyl, arylalkylsulfinyl, heteroarylsulfonyl, heteroarylsulfinyl, heteroarylalkylsulfonyl, heteroarylalkylsulfinyl, (C₁-C₆)-alkylsulfonamido, N,N′-(C₁-C₆)-dialkylsulfonamido, aryl, arylalkyl, aryloxy, arylalkoxy, arylthio, arylalkylthio, heteroaryl, heteroarylalkyl, heteroaryloxy, heteroarylalkoxy, heteroarylthio, heteroarylalkylthio, heterocycloalkyl, and heterocycloalkoxy, any of which may be optionally substituted.

Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and Ru, together with the atoms to which they are attached, may be joined to form an optionally substituted 5- to 6-membered heterocycloalkyl ring; any of which may be optionally substituted.

In other embodiments, methods of use and pharmaceutical compositions of the class of compounds of the present invention have structural Formula I wherein R₂ is selected from the group consisting of hydroxy and Q—C(═O)O—, which may be optionally substituted.

R₁ and R₃ are independently selected from the group consisting of hydrogen, duterium, F, Cl, (C₁-C₃)-alkyl, and (C₁-C₄)-alkoxy, any of which may be optionally substituted and R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted.

R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and R₁₁, together with the atoms to which they are attached, may be joined to form an optionally substituted 5- to 6-membered heterocycloalkyl ring; any of which may be optionally substituted.

In other embodiments, methods of use and pharmaceutical compositions of the class of compounds of the present invention have structural Formula I wherein R₂ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

R₁ and R₃ are independently selected from the group consisting of hydrogen, duterium, F, Cl, (C₁-C₃)-alkyl, and (C₁-C₄)-alkoxy, any of which may be optionally substituted and each of R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted.

R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

In further embodiments, methods of use and pharmaceutical compositions of the class of compounds of the present invention have structural Formula I wherein R₂ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

R₁ and R₃ are independently selected from the group consisting of hydrogen, F, Cl, methyl, ethyl, isobutyl, methoxy, ethoxy, and isopropoxy, any of which may be optionally substituted.

R₄ and R₅ are independently selected from the group consisting of hydrogen, F, Cl, Br, methyl, isopropyl, cyclopropyl and methoxy, any of which may be optionally substituted and substituents R₆ through R₉ are independently selected from the group consisting of hydrogen, CN, NO₂, F, Cl, Br, I, methyl, methoxy, methoxycarbonyl, butoxycarbonyl, amido, ethylamido, N,N-dimethylamido, trifluoromethyl, tetrafluoroethoxy, methylthio, ethylthiomethyl, methylsulfonyl, t-butylsulfinyl, 2-methylsulfonyl-1-ethyl, 2-methylsulfinylmethyl, cyclopropylsulfonyl, cyclopentylsulfinyl, N-propylsulfonamido, and N,N′-diethylsulfonamido, any of which may be optionally substituted.

In still further embodiments, methods of use and pharmaceutical compositions of the class of compounds of the present invention have structural Formula I wherein R₂ is selected from the group consisting of hydroxy and Q, where R₁₀ is methyl and R₁, R₃, and R₄ are each independently selected from the group consisting of hydrogen, F, Cl, methyl, methoxy and ethoxy, any of which may be optionally substituted.

R₅ is hydrogen and R₆ is selected from the group consisting of hydrogen, F, Cl, Br, methyl, and methylsulfonyl. Substituent R₇ is selected from the group consisting of hydrogen, F, Cl, Br, NO₂, methyl, trifluoromethyl, methylsulfonyl and methoxycarbonyl.
R₈ is selected from the group consisting of hydrogen, F, NO₂, Cl, I, methylsulfonyl, methoxycarbonyl, tetrafluoroethoxy, and trifluoromethyl; and R₉ is selected from the group consisting of hydrogen, Cl, methyl, trifluoromethyl, methoxycarbonyl, and NO₂.

In other embodiments, methods of use and pharmaceutical compositions of the class of compounds of the present invention have structural Formula I wherein R₂ is selected from the group consisting of hydroxy and Q, where R₁₀ is methyl.

Substituents R₁ and R₃ through R₅ are hydrogen. R₆ is selected from the group consisting of hydrogen, F, Cl, Br, methyl, and methylsulfonyl and substituent R₇ is selected from the group consisting of hydrogen, F, Cl, Br, NO₂, methyl, trifluoromethyl, methylsulfonyl and methoxycarbonyl.

R₈ is selected from the group consisting of hydrogen, F, NO₂, Cl, I, methylsulfonyl, methoxycarbonyl, tetrafluoroethoxy, and trifluoromethyl; and R₉ is selected from the group consisting of hydrogen, Cl, methyl, trifluoromethyl, methoxycarbonyl, and NO₂.

In yet other embodiments, methods of use and pharmaceutical compositions of the class of compounds of the present invention have structural Formula I wherein R₃ is selected from the group consisting of hydroxy, carboxy, (C₁-C₃)-alkoxycarbonyl, and Q—C(═O)O—, any of which may be optionally substituted.

R₁ and R₂ are independently selected from the group consisting of hydrogen, hydroxy, duterium, F, Cl, Br, (C₁-C₃)-alkyl, (C₃-C₆)-cycloalkyl, (C₁-C₄)-alkoxy, and Q—C(═O)O—, any of which may be optionally substituted; or R₁ and R₂ may be combined together with the atoms to which they are attached and joined to form an optionally substituted 5-to 6-membered heterocycloalkyl ring.

R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted.

Substituent groups R₆ through R₉ are independently selected from the group consisting of hydrogen, CN, NO₂, F, Cl, Br, I, (C₁-C₆)-alkyl, (C₃-C₆)-cycloalkyl, (C₁-C₆)-alkoxy, (C₃-C₆)-cycloalkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, arylsulfonyl, arylsulfinyl, arylalkylsulfonyl, arylalkylsulfinyl, heteroarylsulfonyl, heteroarylsulfinyl, heteroarylalkylsulfonyl, heteroarylalkylsulfinyl, (C₁-C₆)-alkylsulfonamido, N,N′-(C₁-C₆)-dialkylsulfonamido, aryl, arylalkyl, aryloxy, arylalkoxy, arylthio, arylalkylthio, heteroaryl, heteroarylalkyl, heteroaryloxy, heteroarylalkoxy, heteroarylthio, heteroarylalkylthio, heterocycloalkyl, and heterocycloalkoxy, any of which may be optionally substituted.

Q is R₁₀, OR₁₀, NHR₁₀, or NR₁₀R₁₁; R₁₀ and R_{H} are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and R₁₁, together with the atoms to which they are attached, may be joined to form an optionally substituted 5- to 6-membered heterocycloalkyl ring; any of which may be optionally substituted.

In yet other embodiments, methods of use and pharmaceutical compositions of the class of compounds of the present invention have structural Formula I wherein R₃ is selected from the group consisting of hydroxy and Q—C(═O)O—, which may be optionally substituted.

R₁ and R₂ are independently selected from the group consisting of hydrogen, duterium, F, Cl, (C₁-C₃)-alkyl, and (C₁-C₄)-alkoxy, any of which may be optionally substituted.

R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted and R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and R₁₁, together with the atoms to which they are attached, may be joined to form an optionally substituted 5- to 6-membered heterocycloalkyl ring; any of which may be optionally substituted.

In certain embodiments, methods of use and pharmaceutical compositions of the class of compounds of the present invention have structural Formula I wherein R₃ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

R₁ and R₂ are independently selected from the group consisting of hydrogen, duterium, F, Cl, (C₁-C₃)-alkyl, and (C₁-C₄)-alkoxy, any of which may be optionally substituted, and R₄ and R₅ are independently selected from the group consisting of hydrogen, duterium, F, Cl, Br, (C₁-C₃)-alkyl, (C₃-C₆)-cycloalkyl, and (C₁-C₃)-alkoxy, any of which may be optionally substituted.

R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

In certain embodiments, methods of use and pharmaceutical compositions of the class of compounds of the present invention have structural Formula I wherein R₃ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

R₁ and R₂ are independently selected from the group consisting of hydrogen, F, Cl, methyl, ethyl, isobutyl, methoxy, ethoxy, and isopropoxy, any of which may be optionally substituted.

R₄ through R₅ are independently selected from the group consisting of hydrogen, F, Cl, Br, methyl, isopropyl, cyclopropyl and methoxy, any of which may be optionally substituted; and R₆ through R₉ are independently selected from the group consisting of hydrogen, CN, NO₂, F, Cl, Br, I, methyl, methoxy, methoxycarbonyl, butoxycarbonyl, amido, ethylamido, N,N-dimethylamido, trifluoromethyl, tetrafluoroethoxy, methylthio, ethylthiomethyl, methylsulfonyl, t-butylsulfinyl, 2-methylsulfonyl-1-ethyl, 2-methylsulfinylmethyl, cyclopropylsulfonyl, cyclopentylsulfinyl, N-propylsulfonamido, and N,N′-diethylsulfonamido, any of which may be optionally substituted.

In still further embodiments, methods of use and pharmaceutical compositions of the class of compounds of the present invention have structural Formula I wherein R₃ is selected from the group consisting of hydroxy and Q, where R₁₀ is methyl.

R₁, R₂, and R₄ are independently selected from the group consisting of hydrogen, F, Cl, methyl, methoxy and ethoxy, any of which may be optionally substituted and R₅ is hydrogen.

R₆ is selected from the group consisting of hydrogen, F, Cl, Br, methyl, and methylsulfonyl and R₇ is selected from the group consisting of hydrogen, F, Cl, Br, NO₂, methyl, trifluoromethyl, methylsulfonyl and methoxycarbonyl.

Substituent R₈ is selected from the group consisting of hydrogen, F, NO₂, Cl, I, methylsulfonyl, methoxycarbonyl, tetrafluoroethoxy, and trifluoromethyl, while substituent R₉ is selected from the group consisting of hydrogen, Cl, methyl, trifluoromethyl, methoxycarbonyl, and NO₂.

In yet further embodiments, methods of use and pharmaceutical compositions of the class of compounds of the present invention have structural Formula I wherein R₃ is selected from the group consisting of hydroxy and Q, where R₁₀ is methyl. Each of R₁, R₂, R₄ and R₅ are hydrogen and R₆ is selected from the group consisting of hydrogen, F, Cl, Br, methyl, and methylsulfonyl.

R₇ is selected from the group consisting of hydrogen, F, Cl, Br, NO₂, methyl, trifluoromethyl, methylsulfonyl and methoxycarbonyl.

R₈ is selected from the group consisting of hydrogen, F, NO₂, Cl, I, methylsulfonyl, methoxycarbonyl, tetrafluoroethoxy, and trifluoromethyl and R₉ is selected from the group consisting of hydrogen, Cl, methyl, trifluoromethyl, methoxycarbonyl, and NO₂.

Compounds in accordance with the present invention include the following:
N-(2-fluorophenyl)-2-hydroxybenzamide;
N-(4-fluorophenyl)-2-hydroxybenzamide;
N-(3-fluorophenyl)-2-hydroxybenzamide;
N-(2-bromophenyl)-2-hydroxybenzamide;
N-(3-chlorophenyl)-2-hydroxybenzamide;
5-chloro-N-(2-chloro-4-nitrophenyl)-2-hydroxybenzamide;
2-hydroxy-N-(3-nitrophenyl)benzamide;
2-hydroxy-N-(4-nitrophenyl)benzamide;

N-(3-fluorophenyl)-2-hydroxy-3-methylbenzamide;
N-(4-fluorophenyl)-2-hydroxy-3-methylbenzamide;
2-(3-fluorophenylcarbamoyl)benzoic acid;
2-(4-fluorophenylcarbamoyl)benzoic acid;
N-(3-bromophenyl)-2-hydroxybenzamide;
N-(3-chlorophenyl)-2-hydroxy-3-methylbenzamide;
2-(2-chlorophenylcarbamoyl)benzoic acid;
2-(2-fluorophenylcarbamoyl)benzoic acid;
2-hydroxy-3-methyl-N-(3-nitrophenyl)benzamide;
N-(2-chlorophenyl)-2-hydroxy-3-methylbenzamide;
N-(3-bromophenyl)-2-hydroxy-3-methylbenzamide;
N-(4-chlorophenyl)-2-hydroxy-3-methylbenzamide;
N-(2-fluorophenyl)-2-hydroxy-3-methylbenzamide;
N-(4-chlorophenyl)-2-hydroxybenzamide;
N-(2-chlorophenyl)-2-hydroxybenzamide;
2-(3-fluorophenylcarbamoyl)phenyl acetate;
4-chloro-2-(2-chloro-4-nitrophenylcarbamoyl)phenyl acetate;
2-(3-(trifluoromethyl)phenylcarbamoyl)phenyl acetate;
2-hydroxy-N-(3-(trifluoromethyl)phenyl)benzamide;
2-(2-(methylsulfonyl)phenylcarbamoyl)phenyl acetate;
2-hydroxy-N-(2-(methylsulfonyl)phenyl)benzamide;
2-(3-(methylsulfonyl)phenylcarbamoyl)phenyl acetate;
2-hydroxy-N-(3-(methylsulfonyl)phenyl)benzamide;
2-(4-(methylsulfonyl)phenylcarbamoyl)phenyl acetate;
2-hydroxy-N-(4-(methylsulfonyl)phenyl)benzamide;
2-(3,5-bis(trifluoromethyl)phenylcarbamoyl)phenyl acetate;
N-(3,5-bis(trifluoromethyl)phenyl)-2-hydroxybenzamide;
2-(3-bromophenylcarbamoyl)phenyl acetate;
2-(4-fluorophenylcarbamoyl)phenyl acetate;
2-(4-chlorophenylcarbamoyl)phenyl acetate;
2-(4-bromophenylcarbamoyl)phenyl acetate;
2-(4-iodophenylcarbamoyl)phenyl acetate;
N-(2-chloro-5-(trifluoromethyl)phenyl)-2-hydroxybenzamide;
N-(5-chloro-2-methyl-4-nitrophenyl)-2-hydroxybenzamide;
2-(2,3-dimethylphenylcarbamoyl)phenyl acetate;
methyl 4-(2-acetoxybenzamido)benzoate; dimethyl 5-(2-acetoxybenzamido)isophthalate;
2-(3,5-dichlorophenylcarbamoyl)phenyl acetatep;
2-(3,5-dimethylphenylcarbamoyl)phenyl acetate;
2-(3-chloro-4-methylphenylcarbamoyl)phenyl acetate;
2-hydroxy-N-(4-(1,1,2,2-tetrafluoroethoxy)phenyl)benzamide;
N-(4-fluoro-3-nitrophenyl)-2-hydroxybenzamide;
2-hydroxy-N-(4-(trifluoromethyl)phenyl)benzamide;
2-(2-chloro-5-(trifluoromethyl)phenylcarbamoyl)phenyl acetate;
2-(4-chloro-3-nitrophenylcarbamoyl)phenyl acetate;
2-(2-chloro-5-nitrophenylcarbamoyl)phenyl acetate; and 2-(3,4-difluorophenylcarbamoyl)phenyl acetate.

The term “salts” is used in its broadest sense. For example, the term salts includes hydrogen salts and hydroxide salts with ions of the present compound. In some embodiments, the term salt may be a subclass referred to as pharmaceutically acceptable salts, which are salts of the present compounds having a pharmacological activity and which are neither biologically nor otherwise undesirable. In all embodiments, the salts can be formed with acids, such as, without limitation, hydrogen, acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycero-phosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. In all embodiments, the salts can be formed with bases, such as, without limitation, hydroxide, ammonium salts, alkali metal salts such as lithium, sodium and potassium salts, alkaline earth metal salts such as calcium, magnesium salts, aluminum salts, salts with organic bases such as ammonia, methylamine, diethylamine, ethanolamine, dicyclohexylamine, N-methylmorpholine, N-methyl-D-glucamine, and salts with amino acids such as arginine and lysine. Basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy, phenol or similar group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

The term “solvates” is used in its broadest sense. For example, the term solvates includes hydrates formed when a compound of the present invention contains one or more bound water molecules.

As used in the present specification the following terms have the meanings indicated:

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain, branched-chain, and cyclic unsaturated hydrocarbon radical having one or more double bonds and containing from 2 to 20, preferably 2 to 6, carbon atoms. The term “alkenyl groups” is used in its broadest sense. Alkenylene refers to a carbon-carbon double bond system attached at two or more positions such as ethenylene [(—CH═CH—),(—C::C—)]. For example, (C₂-C₈) alkenyl groups embraces straight, branched, and cyclic hydrocarbon chains containing 2 to 8 carbon atoms having at least one double bond, Examples of suitable alkenyl radicals include ethenyl, propenyl, iso-propenyl, butenyl, iso-butenyl, sec-butenyl, tert-butenyl, n-pentenyl, n-hexenyl, and the like, unless otherwise indicated.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. More preferred alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. The term “alkyl groups” is used in its broadest sense. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. For example, the O(C₁-C₈)-alkyl groups comprises the straight O(C₁-C₈)-alkyl groups as well as the branched O(C₁-C₈)-alkyl groups. For another example, the term comprises cycloalkyl groups, as for example, the (C₁-C₈)-alkyl groups comprises the (C₃-C₈)-cycloalkyl groups.

The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an amino group attached to the parent molecular moiety through an alkyl group.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfinyl,” as used herein, alone or in combination, refers to an alkyl group attached to the parent molecular moiety through a sulfinyl group (RSO₂—). Examples of alkylsulfinyl groups include methylsulfinyl, ethylsulfinyl, butylsulfinyl and phenylsulfinyl.

The term “alkylsulfonyl,” as used herein, alone or in combination, refers to an alkyl group attached to the parent molecular moiety through a sulfonyl group (RSO₂—). Examples of alkylsulfonyl groups include methanesulfonyl, ethanesulfonyl, tert-butanesulfonyl, benzylsulfonyl and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein in its broadest sense, alone or in combination, refers to a straight-chain, branched chain hydrocarbon and cyclic unsaturated hydrocarbon radicals having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). For example, (C₂-C₈) alkynyl groups embraces straight, branched, and cyclic hydrocarbon chains containing 2 to 8 carbon atoms having at least one triple bond, and the term includes but is not limited to substituents such as ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like, unless otherwise indicated.

The term “amido,” as used herein, alone or in combination, refers to an amino group as described below attached to the parent molecular moiety through a carbonyl group. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “amino,” as used herein, alone or in combination, refers to —NRR′, wherein R and R′ are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted with one, two, three, four, or five substituents independently selected from the group consisting of alkenyl, alkoxy, alkoxyalkyl, alkyl, cyano, halo, haloalkoxy, haloalkyl, hydroxy, hydroxy-alkyl, nitro, and oxo.

The term “aminoalkyl,” as used herein, alone or in combination, refers to an amino group attached to the parent molecular moiety through an alkyl group. Examples include aminomethyl, aminoethyl and aminobutyl. The term “alkylamino” denotes amino groups which have been substituted with one or two alkyl radicals. Suitable “alkylamino” groups may be mono- or dialkylated, forming groups such as, for example, N-methylamino, N-ethylamino, N,N-dimethylamino, N,N-diethylamino and the like.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an —C(N—CN)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group

The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl-O—C(O)— in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl(hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as phenyl, naphthyl, anthracenyl, phenanthryl, and biphenyl. The aryl groups of the present invention can be optionally substituted with one, two, three, four, or five substituents independently selected from the groups as defined herein.

The term “arylamino” as used herein, alone or in combination, refers to an aryl group attached to the parent moiety through an amino group, such as N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “carboxy” or “carboxyl”, whether used alone or with other terms, such as “carboxyalkyl”, denotes —CO₂H.

The terms “benzo” and “benz,” as used herein, alone or in combination, refer to the divalent radical C₆H₄=derived from benzene. Examples include benzothiophene and benzimidazole.

The term “carbamoyloxy,” as used herein, alone or in combination, refers to an amino-substituted carbonyl group attached to the parent molecular moiety through a oxygen atom (e.g. RR′NC(═O)O—), wherein the amino group can be a primary or secondary amino group containing substituents selected from alkyl, aryl, aralkyl, cycloalkyl, cycloalkylalkyl radicals and the like.

The term “O-carbamyl” as used herein, alone or in combination, refers to a —OC(O)NR, group—with R as defined herein.

The term “C-linked” as used herein, alone or in combination, refers to any substituent that is attached to the parent molecular moiety through a carbon-carbon bond.

The term “N-carbamyl” as used herein, alone or in combination, refers to a ROC(O)NH— group, with R as defined herein.

The term “carbonate” as used herein, alone or in combination, refers to a —O—C(═O)OR group, with R as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” such as a carboxylic acid salt derivative or ester derivative. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably three to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by bicyclo[2,2,2]octane, bicyclo[2,2,2]octane, bicyclo[1,1,1]pentane, camphor and bicyclo[3,2,1]octane.

The term “cycloalkenyl,” as used herein, alone or in combination, refers to a partially unsaturated monocyclic, bicyclic or tricyclic radical wherein each cyclic moiety contains from 3 to 12, preferably five to eight, carbon atom ring members and which may optionally be a benzo fused ring system which is optionally substituted as defined herein. Examples of such cycloalkenyl radicals include cyclopentenyl, cyclohexenyl, cyclohexadienyl, cycloheptenyl, cyclooctadienyl, -1H-indenyl and the like.

The term “cycloalkylalkyl,” as used herein, alone or in combination, refers to an alkyl radical as defined above which is substituted by a cycloalkyl radical as defined above. Examples of such cycloalkylalkyl radicals include cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, cyclohexylbutyl and the like.

The term “cycloalkenylalkyl,” as used herein, alone or in combination, refers to an alkyl radical as defined above which is substituted by a cycloalkenyl radical as defined above. Examples of such cycloalkenylalkyl radicals include 1-methylcyclohex-1-enyl-, 4-ethylcyclohex-1-enyl-, 1-butylcyclopent-1-enyl-, 3-methylcyclopent-1-enyl- and the like.

The term “ester,” as used herein, alone or in combination, refers to a carbonyloxy-(C═O)O— group bridging two moieties linked at carbon atoms. Examples include ethyl benzoate, n-butyl cinnamate, phenyl acetate and the like.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, trichloroethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (—CFH—), difluoromethylene (—CF₂—), chloromethylene (—CHCl—) and the like. Examples of such haloalkyl radicals include chloromethyl, 1-bromoethyl, fluoromethyl, difluoromethyl, trifluoromethyl, 1,1,1-trifluoroethyl, perfluorodecyl and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH2-NH—OCH3.

The term “heteroaryl,” as used herein, alone or in combination, refers to an aromatic five- or six-membered ring, where at least one atom is selected from the group consisting of N, O, and S, and the remaining ring atoms are carbon. The five-membered rings have two double bonds, and the six-membered rings have three double bonds. The heteroaryl groups are connected to the parent molecular group through a substitutable carbon or nitrogen atom in the ring. The term “heteroaryl” also includes systems where a heteroaryl ring is fused to an aryl group, as defined herein, a heterocycle group, as defined herein, or an additional heteroaryl group. Heteroaryls are exemplified by benzothienyl, benzoxazolyl, benzofuranyl, benzimidazolyl, benzthiazolyl benzotriazolyl, cinnolinyl, furyl, imidazolyl, triazolyl [e.g., 4H-1,2,4-triazolyl, 1H-1,2,3-triazolyl, 2H-1,2,3-triazolyl, etc.], tetrazolyl [e.g. 1H-tetrazolyl, 2H-tetrazolyl, etc.], indazolyl, indolyl, isoxazolyl, isoquinolinyl, isothiazolyl, naphthyridinyl, oxadiazolyl [e.g., 1,2,4-oxadiazolyl, 1,3,4-oxadiazolyl, 1,2,5-oxadiazolyl, etc.], oxazolyl, isoxazolyl, purinyl, thiazolyl, isothiazolyl, thienopyridinyl, thienyl, thiadiazolyl [e.g., 1,2,4-thiadiazolyl, 1,3,4-thiadiazolyl, 1,2,5-thiadiazolyl, etc.], pyridinyl, pyridazinyl, pyrimidinyl, pyrazinyl, pyrazolyl, pyrrolyl, pyrido[2,3-d]pyrimidinyl, pyrrolo[2,3-b]pyridinyl, quinazolinyl, quinolinyl, thieno[2,3-c]pyridinyl, tetrazolyl, triazinyl, and the like. The heteroaryl groups of the present invention can be optionally substituted with one, two, three, four, or five substituents independently selected from the groups as defined herein.

Examples of preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, triazolyl, and isoxazolyl

The term “heteroaralkyl” or “heteroarylalkyl,” as used herein, alone or in combination, refers to a heteroaryl group attached to the parent molecular moiety through an alkyl group.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylalkoxy,” as used herein, alone or in combination, refers to a heteroaryl group attached to the parent molecular moiety through an alkoxy group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylsulfonyl,” as used herein, alone or in combination, refers to a heteroaryl group attached to the parent molecular moiety through a sulfonyl group.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “heterocycloalkenyl,” as used herein, alone or in combination, refers to a heterocycle group attached to the parent molecular moiety through an alkenyl group.

The term “heterocycloalkoxy,” as used herein, alone or in combination, refers to a heterocycle group attached to the parent molecular group through an oxygen atom.

The term “heterocycloalkyl,” as used herein, alone or in combination, refers to an alkyl radical as defined above in which at least one hydrogen atom is replaced by a heterocyclo radical as defined above, such as pyrrolidinylmethyl, tetrahydrothienylmethyl, pyridylmethyl and the like.

The term “heterocycloalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl” as used herein, alone or in combination, refers to a linear or branched alkyl group having one to about ten carbon atoms any one of which may be substituted with one or more hydroxyl radicals. Examples of such radicals include hydroxymethyl, hydroxyethyl, hydroxypropyl, hydroxybutyl and hydroxyhexyl.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′SR— group, where R and R′ are as defined herein.

The term “mercaptomercaptyl” as used herein, alone or in combination, refers to a RSR′ S— group, where R is as defined herein.

The term “mercaptyl” as used herein, alone or in combination, refers to an RS— group, where R is as defined herein.

The term “null” refers to a lone electron pair.

The term “nitro,” as used herein, alone or in combination, refers to —NO₂.

The term “optionally substituted” means the anteceding group may be substituted or unsubstituted. When substituted, the substituents of an “optionally substituted” group may include, without limitation, one or more substituents independently selected from the following groups or designated subsets thereof, alone or in combination: hydrogen, carbonyl, thiocarbonyl, carboxyl, lower alkyl carboxylate, lower alkyl carbonate, lower alkyl carbamate, halogen, hydroxy, amino, amido, cyano, hydrazinyl, hydrazinylcarbonyl, alkylhydrazinyl, dialkylhydrazinyl, arylhydrazinyl, heteroarylhydrazinyl, nitro, oxo, thiol, sulfonic acid, trisubstituted silyl, urea, acyl, lower aryloxy, lower acylamino, lower arylthio, lower alkyl, lower alkylamino, lower dialkylamino, lower alkoxy, lower alkoxyalkyl, lower alkylthio, lower alkylsulfonyl, lower alkenyl, lower alkenylamino, lower dialkenylamino, lower alkenyloxy, lower alkenylthio, lower alkenyl sulfonyl, lower alkynyl, lower alkynylamino, lower dialkynylamino, lower alkynyloxy, lower alkynylthio, lower alkynylsulfonyl, lower cycloalkyl, lower cycloalkyloxy, lower cycloalkylamino, lower cycloalkylthio, lower cycloalkylsulfonyl, lower cycloalkylalkyl, lower cycloalkylalkyloxy, lower cycloalkylalkylamino, lower cycloalkylalkylthio, lower cycloalkylalkylsulfonyl, aryl, aryloxy, arylamino, arylthio, arylsulfonyl, arylalkyl, arylalkyloxy, arylalkylamino, arylalkylthio, arylalkylsulfonyl, heteroaryl, heteroaryloxy, heteroarylamino, heteroarylthio, heteroarylsulfonyl, heteroarylalkyl, heteroarylalkyloxy, heteroarylalkylamino, heteroarylalkylthio, heteroarylalkylsulfonyl, heterocycloalkyl, heterocycloalkyloxy, heterocycloalkylamino, heterocycloalkylthio, heterocycloalkylsulfonyl, lower haloalkyl, lower haloalkenyl, lower haloalkynyl, lower perhaloalkyl, lower perhaloalkoxy and lower haloalkoxy. Two substituents may be joined together to form a fused four-, five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. All pendant aryl, heteroaryl, and heterocyclo moieties can be further optionally substituted with one, two, three, four, or five substituents independently selected from the groups listed above.

The terms “oxy” or “oxa,” as used herein, alone or in combination, refer to —O—.

The term “oxo” as used herein, alone or in combination, refers to a doubly bonded oxygen ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphonate” as used herein, alone or in combination, refers to the —P(═O)(OR)(OR1) group.

The term “phosphinate” as ues herein, alone or in combination, refers to the —P(═O)(R)(OR1) group.

The terms “sulfonate,” “sulfonic acid,” and “sulfonic,” as used herein, alone or in combination, refer the —SO3H group and its anion as the sulfonic acid is used in salt formation.

The term “sulfanyl,” as used herein, alone or in combination, refers to —S and —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NH— group with R as defined herein.

The term “S-sulfonamido” refers to a —S(═O)₂NR2, group, with R as defined herein.

The terms “thia” and “thio,” as used herein, alone or in combination, refer to a —S— group or an thioether (R—S—R′) wherein the oxygen atom is replaced with sulfur. The oxidized derivatives of the thio group, namely sulfinyl (—SO—) and sulfonyl (—SO₂—), are included in the definition of thia and thio.

The term “thioether,” as used herein, alone or in combination, refers to a thio group bridging two moieties linked at carbon atoms.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NH— group, with R as defined herein.

The term “I-thiocarbamyl” refers to a —OC(S)NR, group with R as defined herein.

The term “thiocyanato” refers to a —CNS group.

The term “trihalomethanesulfonamido” refers to a X3CS(O)2NR— group with X is a halogen and R as defined herein.

The term “trihalomethanesulfonyl” refers to a X3CS(O)2- group where X is a halogen.

The term “trihalomethoxy” refers to a X3CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

The term “urea,” as used herein, alone or in combination, refers to —N(R)C(═O)N(R)(R), with R as defined herein.

In any embodiment of the compounds of formula (I), R1 through R5 may be the same, may be different, or some members of R1 through R5 may be the same while the others are different. Any combination is possible.

Examples of compounds of the present invention may include, but are not limited to the following compounds listed in Table 1 below:

TABLE 1
Physical Properties of the Inventive Salicylanilides

| | | | Mol. | Mol. | mp | |
|---|---|---|---|---|---|---|
| No | Molstructure | ChemName | Formula | Wt. | (° C.) | Appearence |
| 1 | 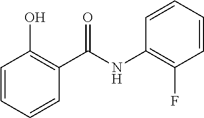 | N-(2-fluorophenyl)-2- hydroxybenzamide | C13H10FNO2 | 231.22 | 146.2 | nearly colorless solid |
| 2 | 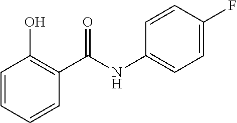 | N-(4-fluorophenyl)-2- hydroxybenzamide | C13H10FNO2 | 231.22 | 163.3 | nearly colorless solid |
| 3 | 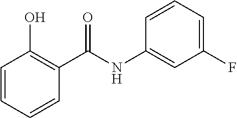 | N-(3-fluorophenyl)-2- hydroxybenzamide | C13H10FNO2 | 231.22 | 157-159 | nearly colorless solid |
| 4 | 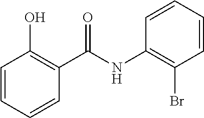 | N-(2-bromophenyl)-2- hydroxybenzamide | C13H10BrNO2 | 292.13 | 165.5 | nearly colorless solid |
| 5 | 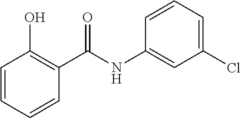 | N-(3-chlorophenyl)-2- hydroxybenzamide | C13H10ClNO2 | 247.68 | 176 | nearly colorless solid |
| 6 | 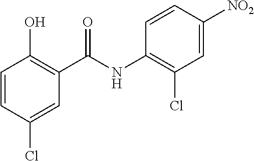 | 5-chloro-N-(2- chloro-4- nitrophenyl)-2- hydroxybenzamide | C13H8Cl2N2O4 | 327.12 | 202-203 | pale yellow solid |
| 7 | 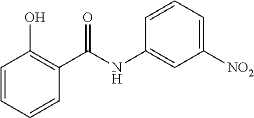 | 2-hydroxy-N-(3- nitrophenyl)benzamide | C13H10N2O4 | 258.23 | 222 | pale yellow solid |
| 8 | 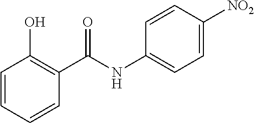 | 2-hydroxy-N-(4- nitrophenyl)benzamide | C13H10N2O4 | 258.23 | 233 | pale yellow solid |
| 9 | 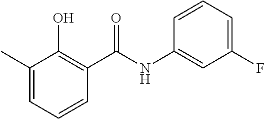 | N-(3-fluorophenyl)-2- hydroxy-3- methylbenzamide | C14H12FNO2 | 245.25 | 143.8-144.8 | nearly colorless solid |
| 10 | 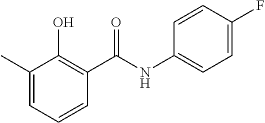 | N-(4-fluorophenyl)-2- hydroxy-3- methylbenzamide | C14H12FNO2 | 245.25 | 144.8-145.8 | nearly colorless solid |
| 11 | 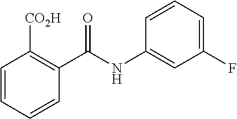 | 2-(3-fluorophenyl- carbamoyl)benzoic acid | C14H10FNO3 | 259.23 | 196-198 | nearly colorless solid |
| 12 | 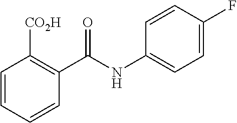 | 2-(4-fluorophenyl- carbamoyl) benzoic acid | C14H10FNO3 | 259.23 | 167.3 | nearly colorless solid |
| 13 | 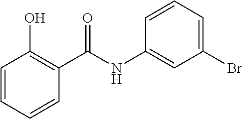 | N-(3-bromophenyl)-2- hydroxybenzamide | C13H10BrNO2 | 292.13 | 183.5 | nearly colorless solid |
| 14 | 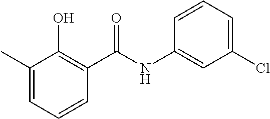 | N-(3-chlorophenyl)-2- hydroxy-3- methylbenzamide | C14H12ClNO2 | 261.70 | 148.5 | nearly colorless solid |
| 15 | 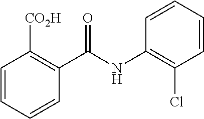 | 2-(2-chloro- phenylcarbamoyl) benzoic acid | C14H10ClNO3 | 275.69 | 145.2-145.7 | nearly colorless solid |
| 16 | 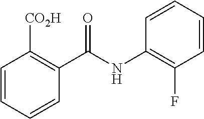 | 2-(2-fluorophenyl- carbamoyl)benzoic acid | C14H10FNO3 | 259.23 | 144.1-144.9 | nearly colorless solid |
| 17 | 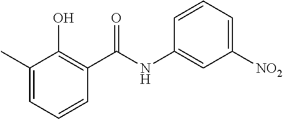 | 2-hydroxy-3-methyl- N-(3-nitrophenyl) benzamide | C14H12N2O4 | 272.26 | 217.5 | pale yellow solid |
| 18 | 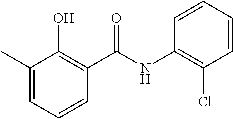 | N-(2-chlorophenyl)-2- hydroxy-3- methylbenzamide | C14H12ClNO2 | 261.70 | 99.8-100.4 | nearly colorless solid |
| 19 | 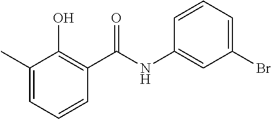 | N-(3-bromophenyl)-2- hydroxy-3- methylbenzamide | C14H12BrNO2 | 306.15 | 147.2-148.2 | nearly colorless solid |
| 20 | 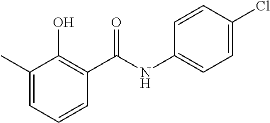 | N-(4-chlorophenyl)-2- hydroxy-3- methylbenzamide | C14H12ClNO2 | 261.70 | 159.6-161.6 | nearly colorless solid |
| 21 | 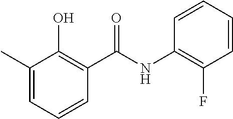 | N-(2-fluorophenyl)-2- hydroxy-3- methylbenzamide | C14H12FNO2 | 245.25 | 74.2-83.4 | nearly colorless solid |
| 22 | 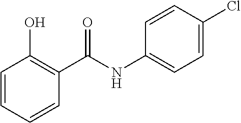 | N-(4-chlorophenyl)-2- hydroxybenzamide | C13H10ClNO2 | 247.68 | 167.1-167.8 | nearly colorless solid |
| 23 | 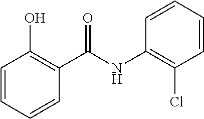 | N-(2-chlorophenyl)-2- hydroxybenzamide | C13H10ClNO2 | 247.68 | 168.1-168.8 | nearly colorless solid |
| 24 | 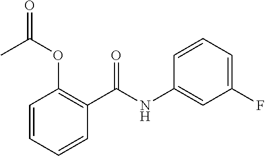 | 2-(3-fluorophenyl- carbamoyl)phenyl acetate | C15H12FNO3 | 273.26 | 88.5-89.7 | colorless solid |
| 25 | 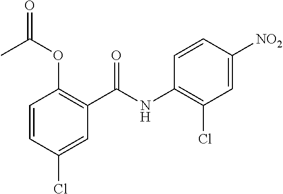 | 4-chloro-2-(2-chloro-4- nitrophenylcarbamoyl) phenyl acetate | C15H10Cl2N2O5 | 369.16 | 179.3-180.5 | very pale yellow solid |
| 26 | 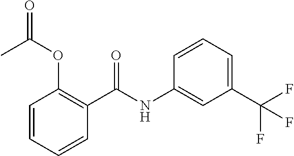 | 2-(3- (trifluoromethyl)phenyl- carbamoyl)phenyl acetate | C16H12F3NO3 | 323.27 | ND | colorless glass |
| 27 | 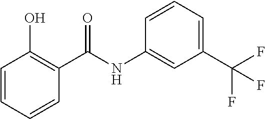 | 2-hydroxy-N-(3- (trifluoromethyl)phenyl) benzamide | C14H10F3NO2 | 281.23 | 188.0-190.6 | colorless solid |
| 28 | 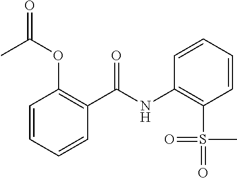 | 2-(2- (methylsulfonyl)phenyl- carbamoyl)phenyl acetate | C16H15NO5S | 333.36 | 115-117 | colorless solid |
| 29 | 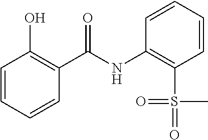 | 2-hydroxy-N-(2- (methylsulfonyl)phenyl) benzamide | C14H13NO4S | 291.32 | 142-145 | colorless solid |
| 30 | 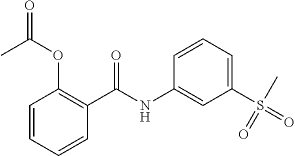 | 2-(3- (methylsulfonyl)phenyl- carbamoyl)phenyl acetate | C16H15NO5S | 333.36 | 51-53; amor- phous | amorphous glass |
| 31 | 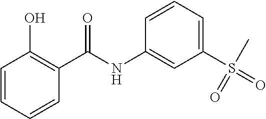 | 2-hydroxy-N-(3- (methylsulfonyl)phenyl) benzamide | C14H13NO4S | 291.32 | 210-212 | colorless solid |
| 32 | 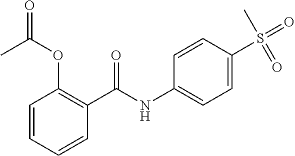 | 2-(4- (methylsulfonyl)phenyl- carbamoyl)phenyl acetate | C16H15NO5S | 333.36 | 184-186 | grey solid |
| 33 | 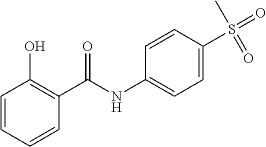 | 2-hydroxy-N-(4- (methylsulfonyl)phenyl)- benzamide | C14H13NO4S | 291.32 | 193-196 | colorless solid |
| 34 | 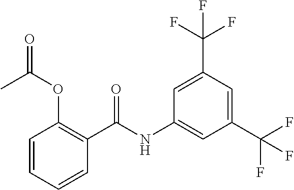 | 2-(3,5- bis(trifluoromethyl) phenylcarbamoyl)phenyl acetate | C17H11F6NO3 | 391.26 | 118-125 | colorless solid |
| 35 | 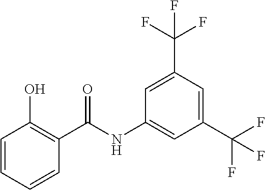 | N-(3,5- bis(trifluoromethyl) phenyl)-2-hydroxy- benzamide | C15H9F6NO2 | 349.23 | 196-198 | colorless solid |
| 36 | 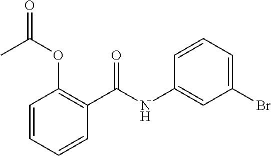 | 2-(3-bromo- phenylcarbamoyl) phenyl acetate | C15H12BrNO3 | 334.16 | ND | ivory solid |
| 37 | 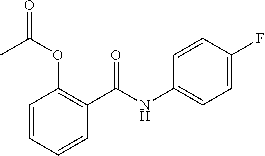 | 2-(4- fluorophenylcarbamoyl) phenyl acetate | C15H12FNO3 | 273.26 | ND | light brown solid |
| 38 | 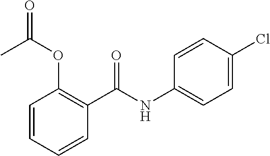 | 2-(4- chlorophenylcarbamoyl) phenyl acetate | C15H12ClNO3 | 289.71 | ND | colorless solid |
| 39 | 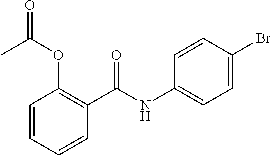 | 2-(4- bromophenylcarbamoyl) phenyl acetate | C15H12BrNO3 | 334.16 | ND | ivory solid |
| 40 | 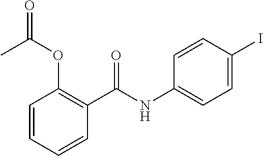 | 2-(4- iodophenylcarbamoyl) phenyl acetate | C15H12NIO3 | 381.17 | ND | pale grey- brown solid |
| 41 | 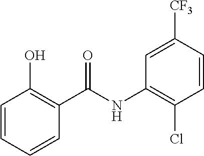 | N-(2-chloro-5- (trifluoromethyl)phenyl)- 2-hydroxybenzamide | C14H9ClF3NO2 | 315.67 | ND | tan solid |
| 42 | 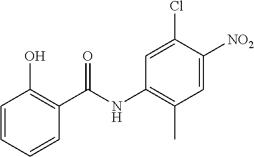 | N-(5-chloro-2-methyl- 4-nitrophenyl)-2- hydroxybenzamide | C14H11ClN2O4 | 306.70 | ND | brown solid |
| 43 | 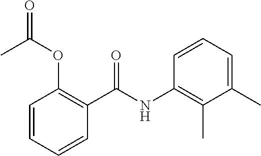 | 2-(2,3-dimethyl- phenylcarbamoyl) phenyl acetate | C17H17NO3 | 283.32 | ND | ivory solid |
| 44 | 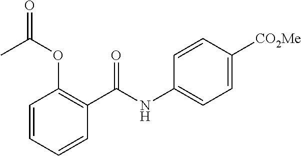 | methyl 4-(2- acetoxybenzamido) benzoate | C17H15NO5 | 313.30 | ND | ivory solid |
| 45 | 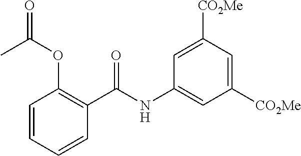 | dimethyl 5-(2- acetoxybenzamido) isophthalate | C19H17NO7 | 371.34 | ND | colorless solid |
| 46 | 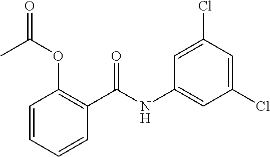 | 2-(3,5-dichloro- phenylcarbamoyl) phenyl acetate | C15H11C12NO3 | 324.16 | ND | ivory solid |
| 47 | 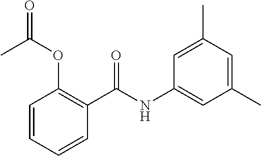 | 2-(3,5-dimethyl- phenylcarbamoyl) phenyl acetate | C17H17NO3 | 283.12 | ND | off-white solid |
| 48 | 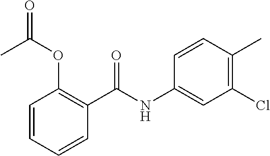 | 2-(3-chloro-4-methyl- phenylcarbamoyl) phenyl acetate | C16H14ClNO3 | 303.74 | ND | ivory white solid |
| 49 | 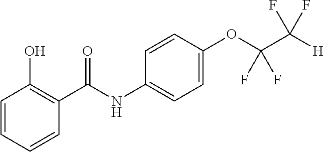 | 2-hyroxy-N-(4- (1,1,2,2-tetra- fluoroethoxy)phenyl) benzamide | C15H11F4NO3 | 329.25 | ND | blue-grey solid |
| 50 | 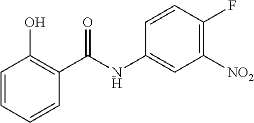 | N-(4-fluoro-3- nitrophenyl)-2- hydroxybenzamide | C13H9FN2O4 | 276.22 | ND | brown solid |
| 51 | 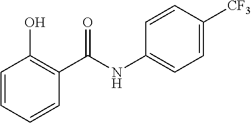 | 2-hydroxy-N-(4- (trifluoromethyl) phenyl)benzamide | C14H10F3NO2 | 281.23 | ND | light yellow solid |
| 52 | 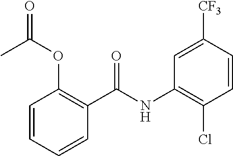 | 2-(2-chloro-5- (trifluoromethyl)phenyl- carbamoyl)phenyl acetate | C16H11ClF3NO3 | 357.71 | ND | light tan solid |
| 53 | 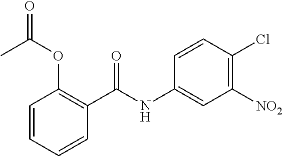 | 2-(4-chloro-3- nitrophenylcarbamoyl) phenyl acetate | C15H11ClN2O5 | 334.71 | ND | golden brown solid |
| 54 | 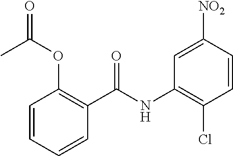 | 2-(2-chloro-5- nitrophenylcarbamoyl) phenyl acetate | C15H11ClN2O5 | 334.71 | ND | very pale yellow solid |
| 55 | 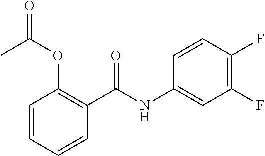 | 2-(3,4-difluoro- phenylcarbamoyl) phenyl acetate | C15H11F2NO3 | 291.25 | ND | pale tan solid |

A compound of the present invention of structural Formula (I) may be synthesized by reacting an aroyl derivative (100), wherein G₁ is hydroxy, chloro, fluoro, bromo, alkoxy and the like with a aniline derivative (102), wherein R₁ through R₉ are as defined herein, under suitable reaction conditions. Optional subsequent hydrolysis then affords compounds (103), where R₁═OH. In some embodiments, the reaction may be generically represented as follows:

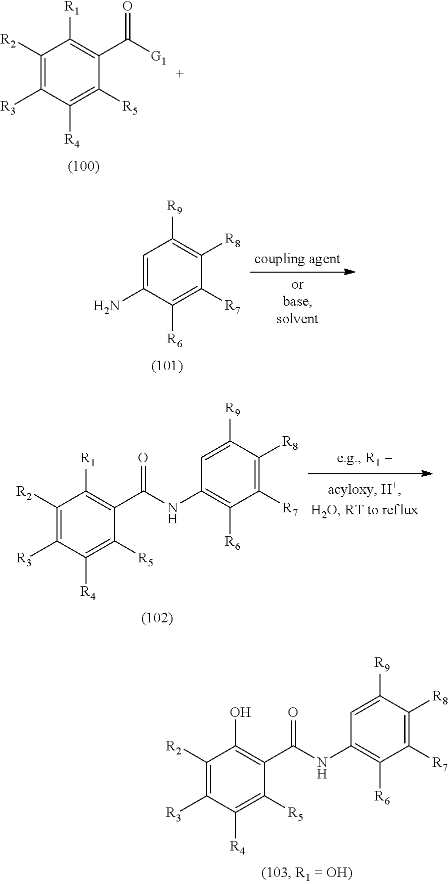

The term carrier is used in its broadest sense. For example, the term carrier refers to any carriers, diluents, excipients, wetting agents, buffering agents, suspending agents, lubricating agents, adjuvants, vehicles, delivery systems, emulsifiers, disintegrants, absorbents, preservatives, surfactants, colorants, flavorants, and sweeteners. In some embodiments, the carrier may be a pharmaceutically acceptable carrier, a term narrower than carrier, because the term pharmaceutically acceptable carrier” means a non-toxic that would be suitable for use in a pharmaceutical composition.

The present invention also relates to a pharmaceutical composition comprising, in a pharmaceutically acceptable carrier, an effective amount of at least one compound of the invention.

The term effective amount is used in its broadest sense. The term, for example, refers to the amount required to produce a desired effect.

In some embodiments, the compound of the invention is present in a pharmaceutical composition in an effective amount for treating HCV infection (e.g., chronic HCV infection). “Treating HCV infection” may refers to: (i) preventing HCV infection from occurring in an animal that may be predisposed to HCV infection but has not yet been diagnosed as having it; (ii) inhibiting or slowing HCV infection, e.g. arresting its development; (iii) relieving chronic infection, e.g. causing its regression; (iv) improving a symptom in a subject having chronic infection; and/or (v) prolonging the survival of a subject having chronic infection.

The compositions of the present invention may be formulated as solid or liquid dosage forms, or as pastes or ointments, and may optionally contain further active ingredients.

A pharmaceutical composition of the present invention comprises a pharmaceutically acceptable carrier, which is not particularly limited, and includes a wide range of carriers known to those of ordinary skill in the art, and including wetting or dispersing agents (U.S. Pat. No. 5,578,621, which is incorporated herein by reference), starch derivatives (U.S. Pat. No. 5,578,621, which is incorporated herein by reference), excipients, and the like. Tablet embodiments may optionally comprise a coating of a substance that constitutes an enteric coating, i.e., a coating that substantially insoluble in gastric secretion but substantially soluble in intestinal fluids.

Pharmaceutical compositions comprising the compounds of the present invention are in some embodiments formulated for oral administration and are optionally in the form of a liquid, for example an emulsion or a solution or a suspension in water or oil such as arachis oil, or other liquid. Formulations of non-aqueous micellar solutions may be prepared according to the method disclosed in U.S. Pat. No. 5,169,846, which is incorporated herein by reference. Alternatively, tablets can be manufactured, for example, by performing the following steps: wet granulation; drying; and compression. Film coating may generally be performed with organic solvents.

The present invention is a method, comprising administering to a subject at least one compound of the present invention in an amount in an effective amount for treating HCV infection (e.g., chronic HCV infection). In some embodiments, the method, comprising administering to a subject at least one pharmaceutical composition which comprises at least one compound of the present invention in an amount in an effective amount for treating HCV infection (e.g., chronic HCV infection).

The present invention is a method, comprising administering to a subject at least one compound of the present invention in an amount in an effective amount for treating HBV infection (e.g., chronic HBV infection). In some embodiments, the method, comprising administering to a subject at least one pharmaceutical composition which comprises at least one compound of the present invention in an amount in an effective amount for treating HBV infection (e.g., chronic HBV infection).

In some embodiments, the subject is chosen from animals. In some embodiments, the subject is chosen from mammals. In some embodiments, the subject is chosen from pets, such as mice, dogs, cats, etc. In some embodiments, the subject is chosen from humans.

In some embodiments, the invention provides a method of treating a viral infection in a subject, comprising administering to the subject at least one dose of an effective amount of at least one compound of the present invention. In some embodiments, the invention provides a method of treating a viral infection in a subject, comprising administering to the subject at least one dose of an effective amount of at least one pharmaceutical composition comprising, in a pharmaceutically acceptable carrier, at least one compound of the present invention.

In some embodiments the antiviral treatment or prophylactic dosages of the compound of the present invention may depend upon the weight of the subject, and may be inferred by one of ordinary skill without undue experimentation by reference to the following examples, which are set forth for purposes of illustration and are not intended to be limiting.

The inventive compounds and compositions may be administered locally or systemically by any means known to an ordinarily skilled artisan. For example, the inventive compounds and compositions may be administered orally, parenterally, by inhalation spray, topically, rectally, nasally, buccally, vaginally or via an implanted reservoir in dosage formulations containing conventional non-toxic pharmaceutically acceptable carriers, adjuvants and vehicles. The term parenteral as used herein includes subcutaneous, intravenous, intraarterial, intramuscular, intraperitoneal, intrathecal, intraventricular, intrasternal, intracranial or intraosseous injection and infusion techniques. The exact administration protocol will vary depending upon various factors including the age, body weight, general health, sex and diet of the patient; the determination of specific administration procedures would be routine to an ordinarily skilled artisan.

Dose levels on the order of about 0.1 to about 100 mg/kg of the active ingredient compound are useful in the treatment of the above conditions (e.g., 0.1 mg/kg-day). In some embodiments, the amounts range from about 1 to about 10 mg/kg, and in other embodiments, the amounts range from about 2 to about 5 mg/kg. The specific dose level for any particular patient will vary depending upon a variety of factors, including the activity and the possible toxicity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

Any administration regimen for regulating the timing and sequence of drug delivery can be used and repeated as necessary to effect treatment. Such regimen may include multiple uses or preadministration and/or co-administration and/or postadministration with food, liquid, or water.

The present invention also relates to a kit, comprising, in a compartment, at least one pharmaceutical composition comprising, in a pharmaceutically acceptable carrier, an effective amount of at least one compound of the invention. In some embodiments, the kit further comprises written instructions for administering the pharmaceutical composition. In some embodiments, written instructions for administering concern indications noted elsewhere in this disclosure. In some embodiments, written instructions for administering concern an administration regimen noted elsewhere in this disclosure.

The kit could take any form. By way of example, a kit includes one or more containers for storing a pharmaceutical composition. In some embodiments, a container contains written instructions for administering the pharmaceutical composition. In some embodiments, a container contains is the substrate for the written instructions for administering the pharmaceutical composition. In some embodiments, the written instructions for administering the pharmaceutical composition are affixed to a container, for example, as in a container for filling a prescription sometimes has written instructions affixed on a surface.

In some embodiments, the compound of the present invention may exhibit selective antiviral activity. The term “selective antiviral” as used herein means that, at dosages effective for the prevention or treatment of a viral disease, the activity is more antiviral than antibacterial, antifungal, or antiparasite, and gut flora of the subject is not disrupted to levels expected with broad spectrum antibiotics. For example, the effective dosage for antiviral treatment (e.g., reducing viral load at least about 2 times) may not reduce bacterial, fungal, or parasite levels in the gut (e.g., more than about 2 times)

Other embodiments of the invention will be apparent to those skilled in the art from consideration of the specification and practice of the invention disclosed herein. It is intended that the specification and its examples be considered as exemplary only, with a true scope and spirit of the invention being indicated by what may eventually be claimed.

### EXPERIMENTAL PROCEDURES

### 1. Materials and Methods

### 1.1 Materials.

All test compounds were provided by Romark Laboratories. Nitazoxanide and Tizoxanide were used as standards. All compounds were dissolved in dimethylsulfoxide (DMSO) stock solution and then diluted using standard serial dilution methods.

### 1.2. HBV Studies.

### 1.2.1. Antiviral Assays.

HBV antiviral assays were conducted as previously described [Korba, B. E. et al. Antiviral Res. 77, 56-63 (2008);]. Briefly, confluent cultures of 2.2.15 cells were maintained on 96-well flat-bottomed tissue culture plates (confluence in this culture system is required for active, high levels of HBV replication equivalent to that observed in chronically-infected individuals. Cultures were treated with nine consecutive daily doses of the test compounds. HBV DNA levels were assessed by quantitative blot hybridization 24 hr. after the last treatment. Cytotoxicity was assessed by uptake of neutral red dye 24 hr. following the last treatment.

### 1.2.3. Production of HBV Proteins.

Cultures of 2.2.15 cells were treated under standard procedures and semi-quantitative EIA-based analysis of HBV proteins was performed as previously described [Korba, B. E. et al. Antiviral Res. 77, 56-63 (2008)]. HBeAg was analyzed ETI-EBK Plus® (DiaSorin, Inc., Stillwater, Minn. USA). Samples were diluted (2 to 10-fold) to bring levels into the dynamic response ranges of the EIA's. HBsAg, and HBeAg were analyzed from culture medium samples and HBcAg was analyzed from intracellular lysates. Intracellular HBV RNA was assessed by quantitative northern blot hybridization.

### 1.3. HCV Studies.

### 1.3.1. Replicon Genotype 1b and 1a Cell Assays.

Antiviral activity of test compounds was assessed in a 3-day assay using the stably-expressing HCV replicon cell line, AVA5 (sub-genomic CONI, genotype 1b) [Korba, B. E. et al. Antiviral Res. 77, 56-63 (2008); Blight et al., Science 290, 1972-1974 (2000)] maintained as sub-confluent cultures on 96-well plates as previously described [Okuse et al., Antiviral Research 65, 23-34 (2005)]. Antiviral activity was determined by blot hybridization analysis of intracellular HCV RNA (normalized to the level of cellular B-actin RNA in each culture sample) and cytotoxicity was assessed by neutral red dye uptake after 3 days of treatment. Additional studies were performed using Huh7 cells containing another HCV replicon, H/FL-Neo, a genotype la full length construct [Blight et al., J. Virol. 77, 3181-3190 (2003)]. For studies involving human serum, standard culture medium (which contains 10% fetal bovine serum) and assay conditions were maintained.

### 1.3.2. Infectious Genotype 2a Cell Culture Assay.

Genotype 2a antiviral assays were conducted as previously described [Broering R. et al., Gut, 59, 1111-1119 (2010)]. In vitro, the Huh7.5/JFH-1 HCV continuous cell culture system was used to study the effects of serial concentrations of NTZ and salicylanilide analogs of the present invention on HCV replication. Cell viability was determined using the Calcein assay kit (Biotium, Inc. Hayward, Calif., USA). The alternate method for measuring cell cytotoxicity was via the Guava Technologies ViaCount assay (Millipore) The ViaCount Assay provides rapid and reliable determinations of viability and total cell count. It distinguishes viable and non-viable cells based on differential permeabilities of two DNA-binding dyes in the Guava ViaCount® Reagent. The nuclear dye stains only nucleated cells, while the viability dye brightly stains dying cells. This combination of dyes enables the Guava ViaCount Assay to distinguish viable, apoptotic, and dead cells. Debris is excluded from results based on negative staining with the nuclear dye.

HCV replication levels were determined by RT-PCR on RNA from cell culture supernatants. J6-infected Huh 7.5 cells were seeded in 12-well plates at a density of 2.0E5 cells/well in 1.5 mL of DMEM supplemented with 10% FBS and 1% Pen/Strep. After overnight incubation, the media was aspirated and replaced with fresh DMEM and NTZ, TIZ, and other thiazolides at concentrations of 1 uM and 10 uM per well to make up a final volume of 1 mL/well. A positive control of IFN-α at concentrations of 10 IU/mL and 50 IU/mL, negative control wells containing mock incubations of DMEM with 1 uL and 10 uL (the same volume of the molecular compounds at 1 uM and 10 uM, respectively) of DMSO was included to rule out nonspecific effects of DMSO on the cells, and J6-infected with only DMEM were used as negative controls. A final control group consisted of un-infected Huh 7.5 cells, plated at the same time and density as the J6-infected cells on a separate plate. 48 hours and 120 hours after incubation with thiazolides, the media was collected and frozen, cells were washed 2× with PBS, and lysed in 500 uL of lysis buffer per well. Following lysis, RNA was extracted from the cells using the RNAqueous-4PCR Kit from Ambion, and the RNA concentration determined using the Nanoprop® machine. Reverse-transcription reactions produced 1.5 μg of cDNA per sample, which was then used for rt-PCR to quantify the HCV viral load in each sample. Analysis of the rt-PCR results generated the fold increases and decreases of the HCV viral load in comparison to untreated J6 cells. The Huh 7.5 controls did not show any HCV virus, as expected.

NTZ and salicylanilide analogs described herein demonstrated efficacy with regards to HCV replication without being cytotoxic to Huh 7.5 cells.

### 1.4. Presentation of Results.

EC50, EC90 and CC50 values (±standard deviations [S.D.]) were calculated by linear regression analysis using data combined from all treated cultures (Korba, B. E. et al. Antiviral Res. 77, 56-63 (2008)). EC50 and EC90 are drug concentrations at which a 2-fold, or a 10-fold depression of intracellular HBV DNA or HCV RNA (relative to the average levels in untreated cultures), respectively, was observed. CC50 is the drug concentration at which a 2-fold lower level of neutral red dye uptake (relative to the average levels in untreated cultures) was observed. Selectivity index (S.I.) was calculated as CC50/EC90 for HBV assays and CC50/EC50 for HCV assays. EC90 values were used for calculation of the S.I. in HBV assays since at least a 3-fold depression of HBV DNA levels is typically required to achieve statistical significance in this assay system [Korba, B. E. et al. Antiviral Res. 77, 56-63 (2008)].

### 1.5 Synthetic Procedures.

Compounds (I) of the present invention may be synthesized according to the general scheme below, wherein R₁-R₉ are as defined herein. In the first synthetic route compound (I) is prepared by reaction of the aroyl derivative (100), wherein G₁ is hydroxy, chloro, fluoro, bromo, alkoxy and the like, with aniline derivative (101), under suitable coupling conditions, preferably in the presence of a suitable solvent to provide (102). When G₁ is hydroxy, standard coupling agents including DCC, EDC/HOBt, EDC/HOAt and related amide bond-forming reagents may be used to prepare (I). When G₁ is chloro, fluoro, bromo, and alkoxy, tertiary amine bases may be added to prepare (I), including triethylamine, diisopropyethylamine, N-methylpiperidine, N-methymorpholine, DBU, DBN, DABCO, 4-(dimethylamino)pyridine and the like. Alternatively, inorganic bases may be employed including sodium bicarbonate, sodium carbonate, potassium carbonate, cesium carbonate, sodium hydroxide, potassium hydroxide, sodium hydride, potassium hydride and the like. Suitable solvents for all reactions include diethyl ether, tetrahydrofuran, 1,4-dioxane, ethyl acetate, acetonitrile, dichloromethane, dichloroethane, benzene, toluene, pyridine, collidine, lutidine, N,N-dimethylformamide, N,N-dimethylacetamide, N-methyl-2-pyrrolidone, DMSO, water, combinations thereof and the like. Optimal temperature ranges may vary from −25° C. to 250° C. Reactions may optionally be conducted in a microwave reactor at ambient temperature to 250° C.

When R₁=acetoxy or lower acyloxy, hydrolysis of (102) with dilute hydrochoric acid, optionally in the presence of cosolvents like tetrahydrofuran, 1,4-dioxane or acetonitrile at about ambient room temperature to about 50 oC yields the free phenolic compound (103), where R₁=OH.

In the second synthetic route, compound (I) is prepared by reaction of the aroyl derivative (503) with QC(═O)G₁, wherein G1 is as defined above and Q is OR₆, NHR₆, NR₆R₇ or R₈ as defined herein, under suitable coupling conditions, preferably in the presence of a suitable solvent.

In some embodiments, the reaction may be generically represented as follows:

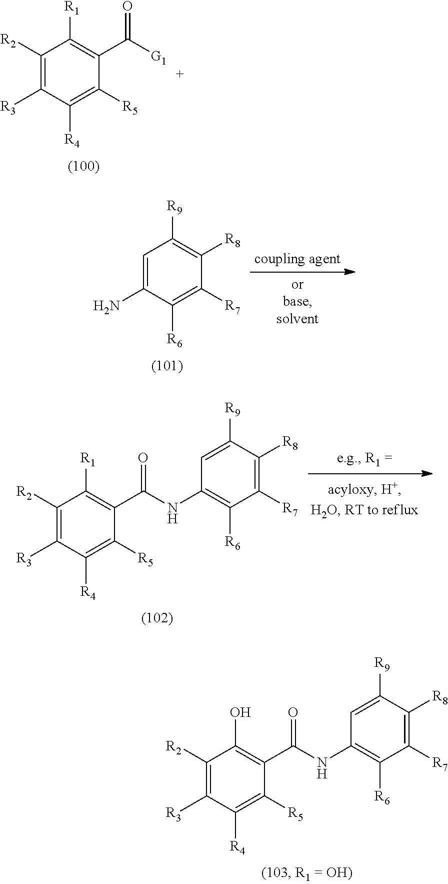

Detailed experimental procedures are described below for compounds 3, 6, 24, and 26-35. By using analogous methods or slight modifications thereof, all remaining compound of the present invention may be prepared.

In addition to the compounds listed in Table 1, further examples of compounds in accordance with the present invention includewithout limitation those illustrated in Table 4.

TABLE 4

| No. | MolStructure |
|---|---|
| 56 | 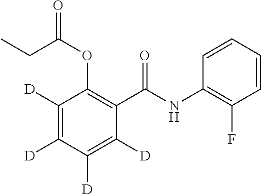 |
| 57 | 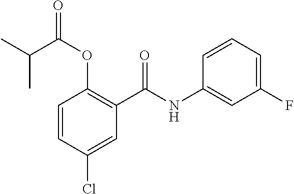 |
| 58 | 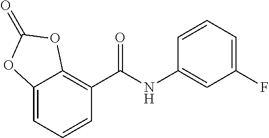 |
| 59 | 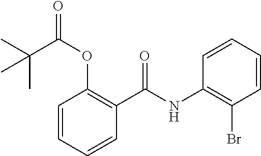 |
| 60 | 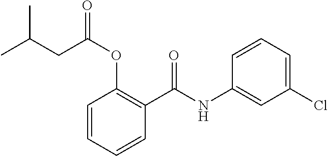 |
| 61 | 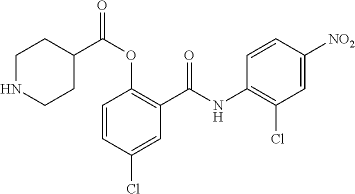 |
| 62 | 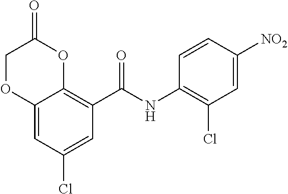 |
| 63 | 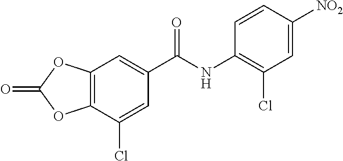 |
| 64 | 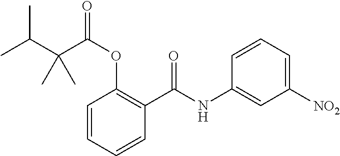 |
| 65 | 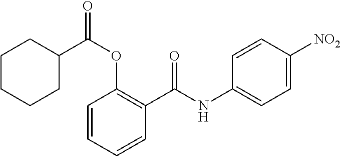 |
| 66 | 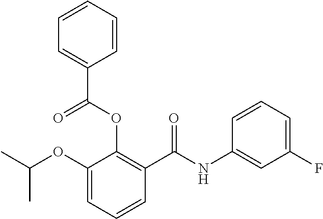 |
| 67 | 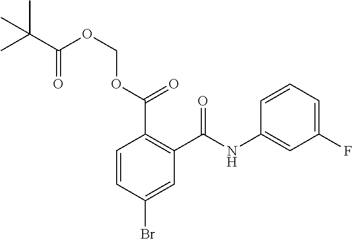 |
| 68 | 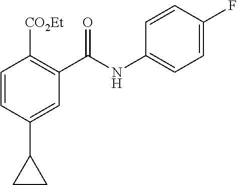 |
| 69 | 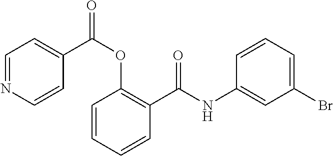 |
| 70 | 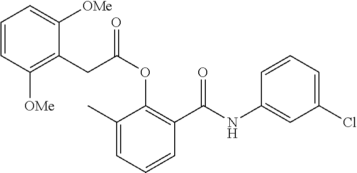 |
| 71 | 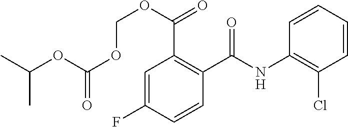 |
| 72 | 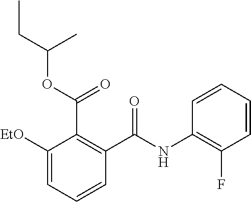 |
| 73 | 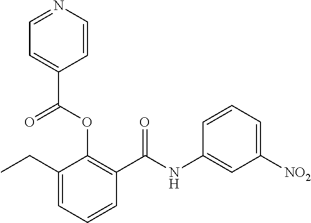 |
| 74 | 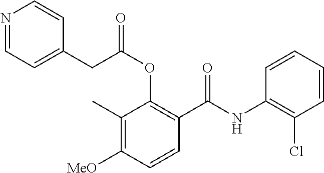 |
| 75 | 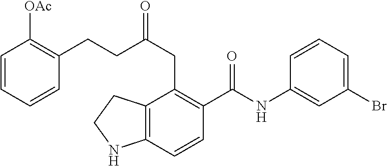 |
| 76 | 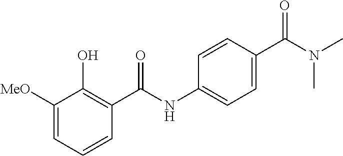 |
| 77 | 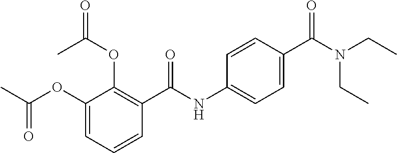 |
| 78 | 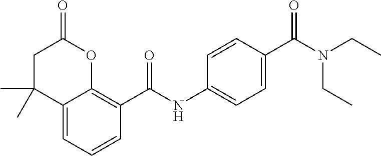 |
| 79 | 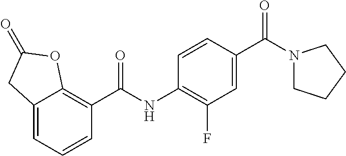 |
| 80 | 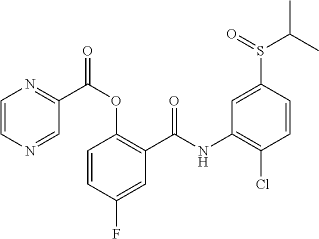 |
| 81 | 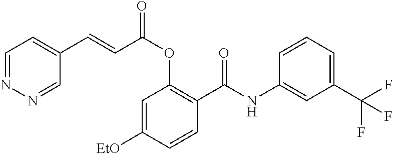 |
| 82 | 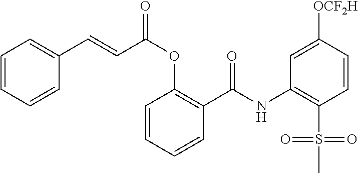 |
| 83 | 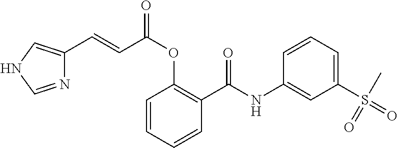 |
| 84 | 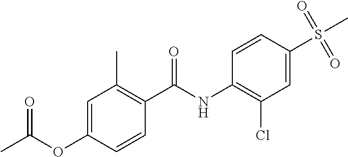 |
| 85 | 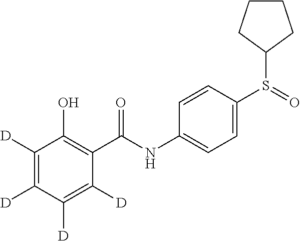 |
| 86 | 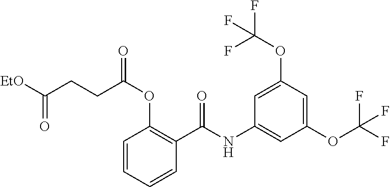 |
| 87 | 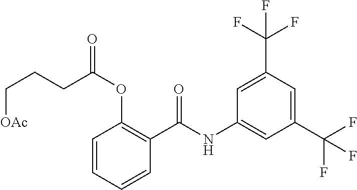 |
| 88 | 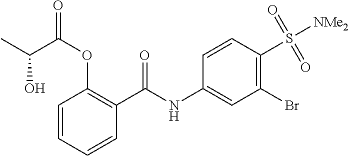 |
| 89 | 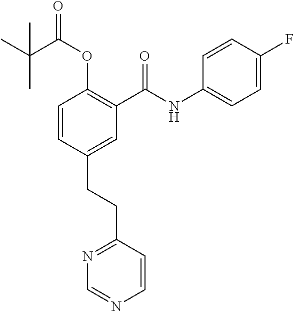 |
| 90 | 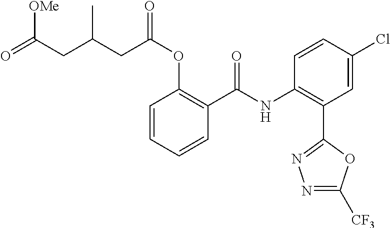 |
| 91 | 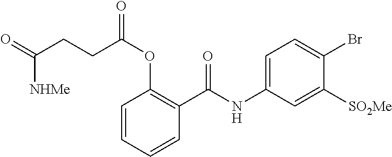 |
| 92 | 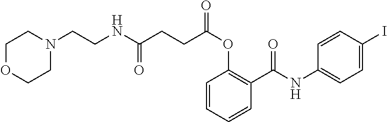 |
| 93 | 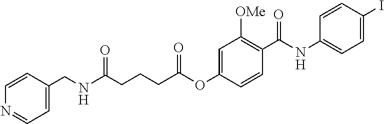 |
| 94 | 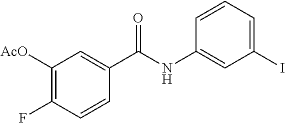 |
| 95 | 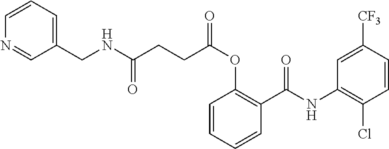 |
| 96 | 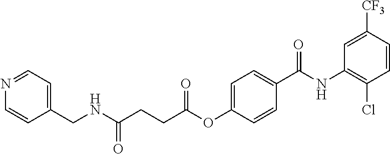 |
| 97 | 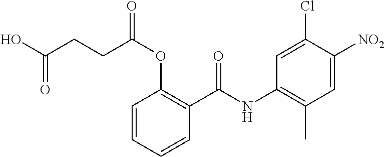 |
| 98 | 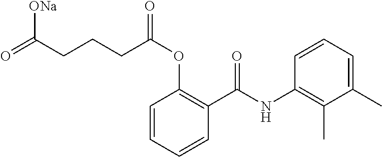 |
| 99 | 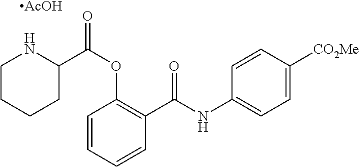 |
| 100 | 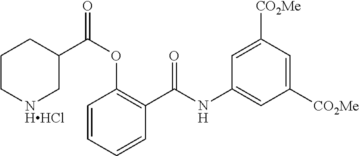 |
| 101 | 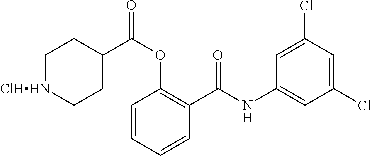 |
| 102 | 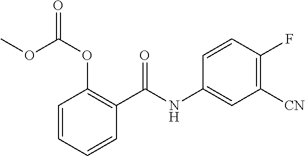 |
| 103 | 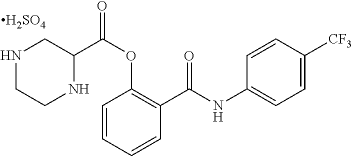 |
| 104 | 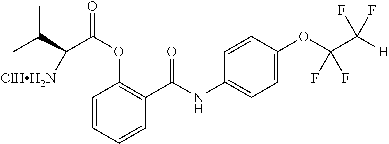 |
| 105 | 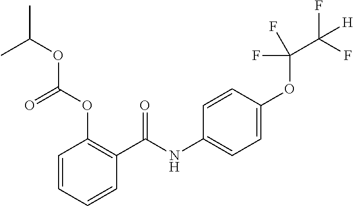 |
| 106 | 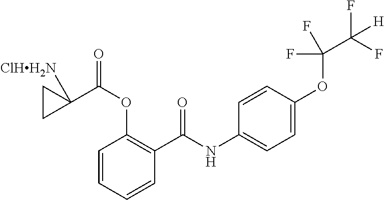 |
| 107 | 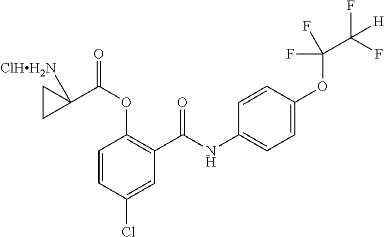 |
| 108 | 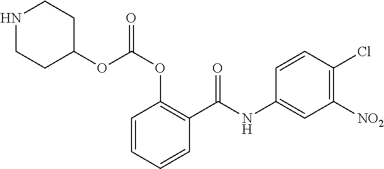 |
| 109 | 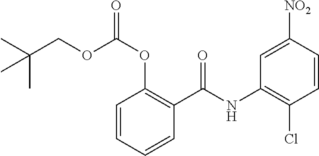 |
| 110 | 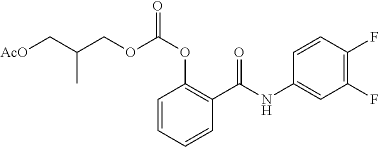 |
| 111 | 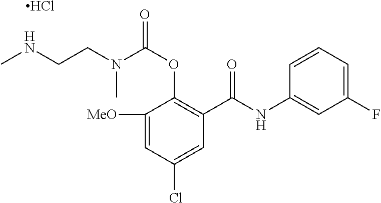 |
| 112 | 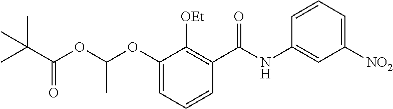 |
| 113 | 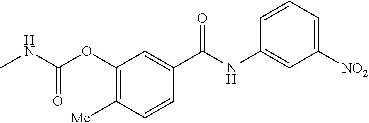 |
| 114 | 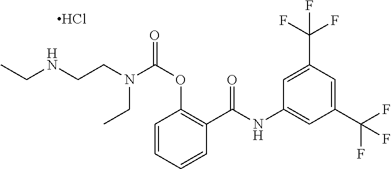 |
| 115 | 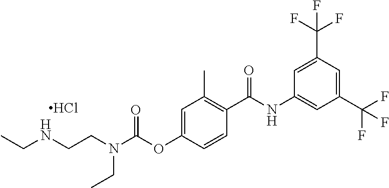 |
| 116 | 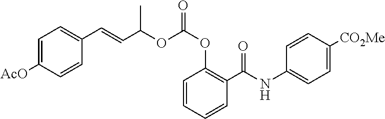 |
| 117 | 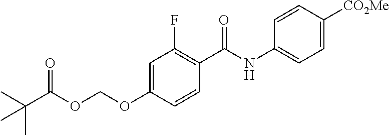 |
| 118 | 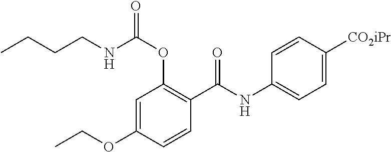 |
| 119 | 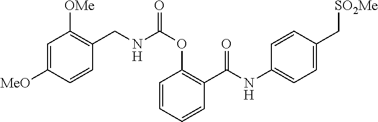 |
| 120 | 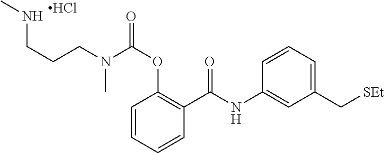 |
| 121 | 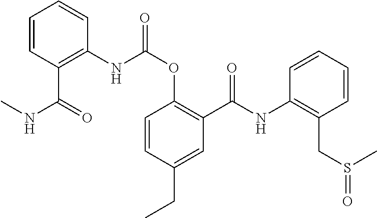 |
| 122 | 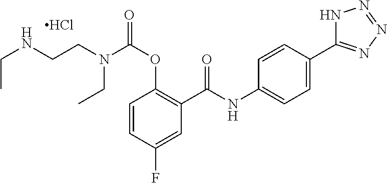 |
| 123 | 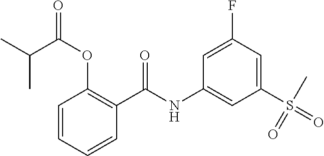 |

### General Experimental Details:

1H NMR spectra were recorded on a Bruker Avance Spectrometer at 400 MHz. 13C NMR spectra were recorded at 100 MHz. Melting points were recorded on a Stanford Research Systems MPA1100 OptiMelt; values are uncorrected. HPLC data was collected on Agilent 1100 HPLC systems with the following columns and conditions:
A: Agilent Zorbax C8 75×4.6 mm 5 micron column (Part #993967-906) maintained at 30° C. Solvent A Water (0.1% TFA); Solvent B Acetonitrile (0.07% TFA), Gradient 5 min 95% A to 95% B; 2 min hold; then recycle; UV Detection @ 210 and 250 nm.
B: Agilent Zorbax Eclipse XDB-C18 50×4.6 mm 1.8 micron column (Part #927975-902) maintained at 30° C. Solvent A Water (0.1% TFA); Solvent B Acetonitrile (0.07% TFA), Gradient 5 min 95% A to 95% B; 1 min hold; 1 min recycle; 30 sec hold. UV Detection @ 210 and 254 nm with no reference.
C: Agilent Zorbax C8 150×4.6 mm 5 micron column maintained at 30° C. Solvent A Water (0.1% TFA); Solvent B Acetonitrile (0.07% TFA), Gradient 10 min 95% A to 95% B; 2 min hold; then recycle. UV Detection @ 210 and 254 nm with no reference.

Flash chromatography was performed with silica gel from Silacycle (40-63 μm, 60 Å) and Whatman (38-63 μm, 60 Å). LC-MS and mass spectra were recorded on a Waters Alliance HT separations module coupled to a Micromass ZMD mass spectrometer.

### 4-Chloro-2-[(2-chloro-4-nitrophenyl)carbamoyl]phenyl acetate (25)

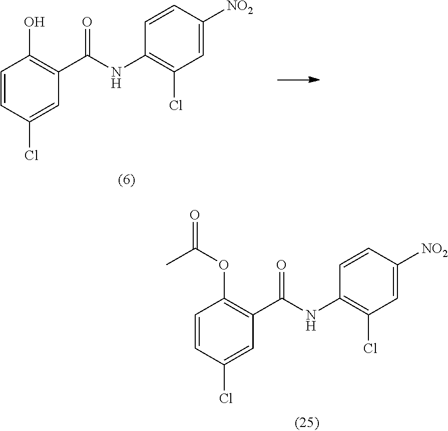

A solution of 5-chloro-N-(2-chloro-4-nitrophenyl)-2-hydroxybenzamide (6, 0.2516 g, 0.7691 mmol) in THF (70 mL) under N2 at room temperature was treated with pyridine (68.0 μL, 0.841 mmol) and acetyl chloride (59.8 μL, 0.841 mmol). The reaction was allowed to stir 6 hours, until complete by HPLC, then filtered and the solvent evaporated in vacuo. The crude product was dissolved in EtOAc (50 mL), then washed with 1M HCl (25 mL) and sat. NaHCO3 (25 mL) before drying over MgSO4 and evaporating the solvent in-vacuo to yield a yellow solid. This crude material was adsorbed onto silica gel with dichloromethane and purified by MPLC (eluting 500 mL each 0, 10, 20, 30, 40% EtOAc:Hexanes). Fractions were pooled and evaporated to give 25 as a very pale yellow solid (162.8 mg, 57%). HPLC tR=8.47 min (99.4%, Condition C). ¹H-NMR (400 MHz, DMSO-d6) δ 10.49 (br s, 1H), 8.42 (d, J=2.7 Hz, 1H), 8.28 (dd, J=8.9, 2.7 Hz, 1H), 8.08 (d, J=8.9 Hz, 1H), 7.85 (d, J=2.5 Hz, 1H), 7.72 (dd, J=8.7, 2.7 Hz, 1H), 7.37 (d, J=8.7 Hz, 1H), 3.01 (s, 3H) ppm. MS (ESI+) m/z 391.0, 393.0 (M+Na)⁺, and (ESI−) 367.1, 369.1 (M−H)−.

General Coupling and Hydrolysis Procedures. Synthesis of 3, 24, and 26-35.

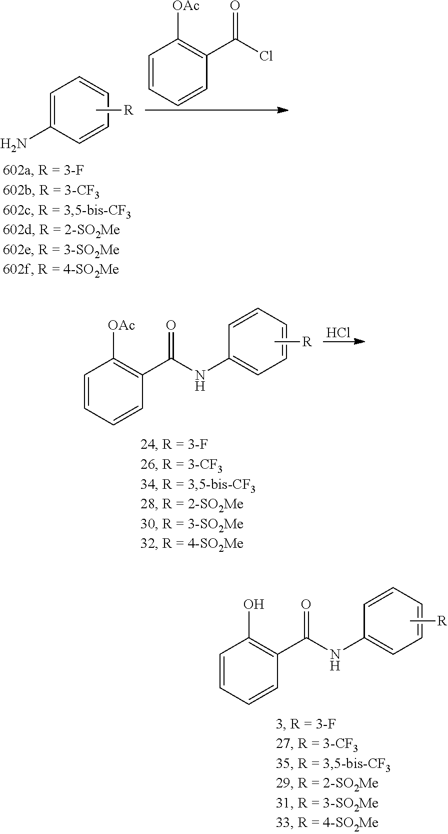

### 2-[(3-Fluorophenyl)carbamoyl]phenyl acetate (24)

3-Fluorobenzenamine (602a, 1.9 mL, 20 mmol) and triethylamine (3.1 mL, 22 mmol, 1.1 equiv) were dissolved in dichloromethane (133 mL) in a 500 mL three-neck RBF equipped with a stir bar, N2 inlet, and an addition funnel with septum. Acetyl-salicyloyl chloride (4.00 g, 20.1 mmol, 1.0 equiv) in dichloromethane (67 mL) was then added in a steady stream from the addition funnel over 8 min, and the reaction was stirred at room temperature for 1 h. The reaction was quenched with HCl (1M, 100 mL), the layers were separated, and the combined organics were washed with [NaHCO3] (2×100 mL), water (2×100 mL), brine (100 mL), dried with MgSO4, filtered, and concentrated in vacuo to a light yellow oil. The crude oil was dissolved in a small amount of EtOAc, and hexanes was added until the solution clouded. This suspension could be concentrated to an off-white solid. The product separated as an oil from attempts to recrystallize from hot EtOAc/hexanes, but crystals would appear in the oil upon standing at room temperature. This mixture could then be swirled vigorously under sonication to give a finely divided colorless solid which was then filtered, washed with hexanes, and dried in vacuo to give 24 (3.47 g, 63%). mp=88.5-89.7° C. (sealed tube). HPLC tR=7.04 min (98.4%, Condition C). ¹H-NMR (400 MHz, DMSO-d6) δ 10.55 (br s, 1H), 7.68 (dd, J=7.8, 1.7 Hz, 1H), 7.65 (app. t, J=2.0 Hz, 1H), 7.59 (ddd, J=7.8, 7.8, 1.7 Hz, 1H), 7.33-7.48 (m, 3H), 7.27 (dd, J=8.3, 1.2 Hz, 1H), 6.93 (dddd, J=8.3, 8.3, 2.9, 1.2 Hz, 1H), and 2.20 (s, 3H) ppm. MS (ESI+) m/z 274.2 (M+H, 296.2 (M+Na)⁺, and (ESI−) 230.2 (M−H)−.

### N-(3-Fluorophenyl)-2-hydroxybenzamide (3)

Compound 24 (5.00 gm, 18.3 mmol) was suspended in conc. aq. hydrochloric acid (50.0 mL) and stirred rapidly. The slurry become homogenous momentarily, and then re-precipitated. The reaction was stirred rapidly at 50° C. for 36 hours before being cooled to room temperature, and filtered on a fine fitted funnel. The solids were washed with water (ca. 50 mL), and dried in vacuo to give 3 (3.97 g, 94%) as a colorless powder without further purification. mp=157.0-159.0° C. (sealed tube). HPLC tR=7.65 min (99.4%, Condition C). ¹H-NMR (400 MHz, DMSO-d6) δ 11.56 (s, 1H), 10.50 (s, 1H), 7.90 (dd, J=7.8, 1.7 Hz, 1H), 7.72 (app. dt, J=12.0, 2.5 Hz, 1H), 7.35-7.50 (m, 3H), and 6.92-7.02 (m, 3H) ppm. MS (ESI+) m/z 232.2 (M+H)⁺, and (ESI−) 230.2 (M−H)−.

### 2-{[3-(Trifluoromethyl)phenyl]carbamoyl}phenyl acetate (26)

3-(Trifluoromethyl)benzenamine (602b, 250 uL, 2.0 mmol) and triethylamine (310 μL, 2.2 mmol, 1.1 equiv) were dissolved in dichloromethane (20 mL) and acetylsalicyloyl chloride (402 mg, 2.0 mmol, 1.0 equiv) was added in a single portion, and the reaction was stirred at room temperature for 1 h. The reaction mixture was quenched with HCl (1 M, 20 mL), the layers were separated, and the organics were washed with [NaHCO3] (20 mL), water (2×20 mL), brine (20 mL), dried with MgSO4, filtered, and concentrated in vacuo to a light yellow oil. The crude oil was filtered through magnesol (eluted 6:1 hexanes/EtOAc) to give 26 (593 mg, 92%) as a colorless glass. HPLC tR=7.86 min (96.8%, Condition C). ¹H-NMR (400 MHz, DMSO-d6) δ 10.68 (s, 1H), 8.17 (br s, 1H), 7.93 (br d, J=8.3 Hz, 1H), 7.72 (dd, J=7.9, 1.7 Hz, 1H), 7.61 (ddd, J=7.5, 7.5, 1.7 Hz, 1H), 7.59 (app. t, J=7.9 Hz), 7.46 (br d, J=7.9 Hz, 1H), 7.42 (ddd, J=7.5, 7.5, 1.0 Hz, 1H), 7.27 (dd, J=8.3, 1.0 Hz, 1H), and 2.20 (s, 3H) ppm. MS (ESI+) m/z 324.1 (M+H)⁺, 346.1 (M+Na)⁺, and (ESI−) 280.2 (M−H)−.

### 2-Hydroxy-N-[3-(trifluoromethyl)phenyl]benzamide (27)

Following the acid hydrolysis procedure reported above for 3 with intermediate 26 gave 27 (234 mg, 91%) as a colorless powder. mp=188.0-190.6° C. (Sealed tube), HPLC tR=8.40 min (98.7%, Condition C). ¹H NMR (400 MHz, DMSO-d6) δ 11.51 (br s, 1H), 10.61 (br s, 1H), 8.23 (s, 1H), 7.95 (br d, J=8.2 Hz), 7.91 (dd, j=7.7, 1.6 Hz, 1H), 7.61 (dd, J=8.0, 8.0, 1H), 7.49 (br d, J=8.0 Hz, 1H), 7.45 (ddd, J=7.5, 7.5, 1.6, 1H), 7.0 (br d, J=8.0 Hz, 1H), and 6.98 (ddd, J=7.5, 7.5, 1.0 Hz, 1H) ppm. MS (ESI+) m/z 282.2 (M+H)⁺, and (ESI−) 280.2 (M−H)⁻.

### 2-{[3,5-Bis(trifluoromethyl)phenyl]carbamoyl}phenyl acetate (34)

Compound 34 was prepared following the procedure discussed above for compound 26 on 2.34 mmol scale with DIPEA (900 mL, 2.2 equiv) as base at room temperature for 21 h. The crude product was recrystallized from hot hexanes/EtOAc (100 mL: 3 mL) to give 34 (266 mg, 29%) as a colorless crystalline solid. The mother liquor contained a significant quantity of 34 but was not pursued. mp=118-125° C. (sealed tube). HPLC tR=8.79 min (99.0%, Condition C). ¹H-NMR (400 MHz, DMSO-d6) δ 10.99 (s, 1H), 8.39 (s, 2H), 7.83 (s, 1H), 7.77 (dd, J=7.5, 1.2 Hz, 1H), 7.64 (ddd, J=7.5, 7.5, 1.6 Hz, 1H), 7.44 (ddd, J=7.5, 7.5, 0.8 Hz, 1H), 7.30 (dd, J=7.5, 0.8 Hz, 1H), and 2.21 (2, 3H) ppm. MS (ESI+) m/z 350.0 (M+H)+, and (ESI−) 348.1 (M−H)−.

### N-[3,5-Bis(trifluoromethyl)phenyl]-2-hydroxybenzamide (35)

Following the procedure reported above for 3 with 34 on 0.40 mmol scale gave 35 (110 mg, 79%) as a colorless powder. mp=196-198° C. (sealed tube). HPLC tR=9.19 min (99.8%, Condition C). ¹H-NMR (400 MHz, DMSO-d6) δ 11.29 (s, 1 H), 10.84 (s, 1H), 8.47 (s, 2H), 7.87 (dd, J=7.8, 1.7 Hz, 1H), 7.84 (s, 1H), 7.46 (app. td, J=7.8, 1.7 Hz, 1H), and 6.95-7.05 (m, 2H) ppm. MS (ESI+) m/z 350.0 (M+H)⁺, and (ESI−) 348.1 (M−H)⁻.

### 2-{[2-(Methylsulfonyl)phenyl]carbamoyl}phenyl acetate (28)

Compound 28 was prepared following the procedure discussed above for compound 26 on a 2.0 mmol scale over 18 h. The crude product was purified via flash chromatography (6:1 to 1:1 gradient of hexanes/EtOAc), followed by recrystallizating from hot hexanes/EtOAc (10 mL:2.5 mL total) to give 28 (238 mg, 35%) as a colorless crystalline solid. mp=115-117° C. (sealed tube). HPLC tR=6.64 min (99.3%, Condition C). ¹H-NMR (400 MHz, DMSO-d6) δ 10.20 (br s, 1H), 8.12 (d, J=7.5 Hz, 1H), 7.96 (dd, J=7.8, 1.2 Hz, 1H), 7.85 (dd, J=7.8, 1.7 Hz, 1H), 7.79 (ddd, J=7.8, 7.8, 1.2 Hz, 1H), 7.64 (ddd, J=7.5, 7.5, 1.7 Hz, 1H), 7.48 (ddd, J=7.5, 7.5, 0.8 Hz, 1H), 7.45 (ddd, J=7.5, 7.5, 0.8 Hz, 1H), 7.29 (dd, J=7.8, 0.8 Hz, 1H), 3.30 (s, 3H), and 2.24 (2, 3H) ppm. MS (ESI+) m/z 356.1 (M+Na)+, and (ESI−) 332.1 (M−H)−.

### 2-Hydroxy-N-[2-(methylsulfonyl)phenyl]benzamide (29)

Following the procedure reported above for 3 with 28 on 0.36 mmol scale gave 29 (92 mg, 88%) as a colorless powder. mp=142-145° C. (sealed tube). HPLC tR=6.78 min (99.3%, Condition C). ¹H NMR (400 MHz, DMSO-d6) δ 11.70 (s, 1H), 11.10, 8.38 (dd, J=8.3, 1.0 Hz, 1H), 7.97 (dd, J=8.0, 1.7 Hz, 1H), 7.94 (dd, J=8.3, 7.4, 1.7 Hz, 1H), 7.76 (ddd, J=7.9, 7.9, 1.7 Hz, 1H), 7.44 (ddd J=8.3, 7.2, 1.7), 7.41 (ddd, J=7.7, 7.7, 1.0 Hz, 1H), 7.02 (dd, J=8.3, 0.8 Hz, 1H), 6.99 (ddd, J=8.0, 7.2, 1.0 Hz, 1H), and 3.24 (s, 3H) ppm. MS (ESI+) m/z 292.2 (M+H)⁺, 314.1 (M+Na)⁺, and (ESI−) 290.2 (M−H)⁻.

### 2-{[3-(Methylsulfonyl)phenyl]carbamoyl}phenyl acetate (30)

Compound 30 was prepared following the procedure discussed above for compound 26 on a 2.0 mmol scale with 3-(methylsulfonyl)aniline hydrochloride and DIPEA (740 μL, 4.2 mmol, 2.1 equiv). The crude product was purified via flash chromatography (4:1 to 1:3 gradient of hexanes/EtOAc) to give 30 (598 mg, 89%) as an amorphous glass. mp=51-53° C. (sealed tube). HPLC tR=5.67 min (96.9%, Condition C). ¹H-NMR (400 MHz, DMSO-d6) δ 10.74 (s, 1H), 8.36 (s, 1H), 7.97 (ddd, J=7.4, 1.7, 1.7 Hz, 1H), 7.73 (dd, J=7.5, 1.7 Hz, 1H), 7.55-7.68 (m, 3H), 7.42 (ddd, J=Hz, 1H), 7.27 (dd, J=7.5, 0.8 Hz, 1H), 3.22 (s, 3H), and 2.21 (s, 3H) ppm. MS (ESI+) m/z 356.0 (M+Na)+.

### 2-Hydroxy-N-[3-(methylsulfonyl)phenyl]benzamide (31)

Following the procedure reported above for 3 with 30 on 0.49 mmol scale gave 31 (120 mg, 85%) as a colorless powder after 19 h at room temperature. mp=210-212° C. (sealed tube, compound undergoes a solid phase transition from 183-191° C.). HPLC tR=6.25 min (99.4%, Condition C). ¹H-NMR (400 MHz, DMSO-d6) δ 11.55 (s, 1H), 10.67 (s, 1H), 8.39 (s, 1H), 8.02 (ddd, J=7.5, 1.7, 1.7 Hz, 1H), 7.93 (dd, J=7.9, 1.7 Hz, 1H), 7.63-7.71 (m, 3H), 7.45 (ddd, J=8.3, 7.5, and 1.7 Hz, 1 H), 7.00 (dd, J=8.3, 0.8 Hz, 1H), 6.98 (ddd, J=8.3, 7.5, 0.8 Hz, 1H), and 3.23 (s, 3H) ppm. MS (ESI+) m/z 292.0 (M+H)⁺, and (ESI−) 290.1 (M−H)⁻.

### 2-{[4-(Methylsulfonyl)phenyl]carbamoyl}phenyl acetate (32)

Compound 32 was prepared following the procedure discussed above for compound 26 on a 2.3 mmol scale with 4-(methylsulfonyl)aniline hydrochloride and DIPEA (890 μL, 5.1 mmol, 2.2 equiv). The crude solids were recrystallized from hot hexanes/EtOAc to give 32 (537 mg, 69%) as a grey crystalline solid. mp=184-186° C. (sealed tube). HPLC tR=5.65 min (97.7%, Condition C). ¹H-NMR (400 MHz, DMSO-d6) δ 10.81 (s, 1H), 7.96 (nfo d, J=9.0 Hz, 2H), 7.90 (d, J=9.0 Hz, 2H), 7.72 (dd, J=7.9, 1.7 Hz, 1H), 7.62 (ddd, J=7.5, 7.5, 1.7 Hz, 1H), 7.43 (ddd, J=7.5, 7.5, 0.8 Hz, 1H), 7.29 (dd, J=7.9, 0.8 Hz, 1H), 3.19 (2, 2H), and 2.20, (s, 3H) ppm. MS (ESI+) m/z 334.0 (M+H)⁺.

### 2-Hydroxy-N-[4-(methylsulfonyl)phenyl]benzamide (33)

Following the procedure reported above for 3 with 32 on 0.82 mmol scale at room temperature for 24 h followed by overnight at 50° C. gave 33 (203 mg, 85%) as a colorless powder. mp=193-193° C. (sealed tube). HPLC tR=6.21 min (99.3%, Condition C). ¹H-NMR (400 MHz, DMSO-d6) δ 11.46 (s, 1H), 10.69 (s, 1H), 7.99 (nfo d, J=9.0 Hz, 2H), 7.91 (nfo d, J=9.0 Hz, 2H), 7.90 (dd, J=7.8, 1.7 Hz, 1H), 7.45 (ddd, J=8.3, 7.3, 1.7 Hz, 1H), 6.95-6.03 (m, 2H), and 3.20 (s, 3H) ppm. MS (ESI+) m/z 292.1 (M+H)⁺, and (ESI−) 290.1 (M−H)−.

### 2. Antiviral Test Results

Against genotype G2a, NTZ, used as a reference inhibitor, and compounds 3 and 44 were all shown to inhibit J6/JFH-1HCV replication at a 1 μM test concentration. At 1 μM, the relative activity of compound 3 was estimated to be 65% of NTZ, while the relative potency of compound 44 was estimated to be 53% of NTZ. None of these compounds showed a cytotoxic effect when tested as described herein in Huh 7.5 cells.

Table 2 presents data from the primary and secondary HCV replicon cell assays. The antiviral activity of the inventive salicylanilides against HCV Genotypes 1b and 1a in cell culture is illustrated below.

TABLE 2

| | | | | | SECONDARY ASSAY, | | | | SECONDARY ASSAY, | | | |
|---|---|---|---|---|---|---|---|---|---|---|---|---|
| | PRIMARY ASSAY | | | | GENOTYPE 1B | | | | GENOTYPE 1A | | | |
| | CC50 | EC50 | EC90 | | CC50 | EC50 | EC90 | | CC50 | EC50 | EC90 | |
| No. | (uM) | (uM) | (uM) | SI | (uM) | (uM) | (uM) | SI | (uM) | (uM) | (uM) | SI |
| NTZ | 31.0 | 0.210 | 0.977 | 148.0 | 35.0 | 0.25 | 0.95 | 143.0 | 49.0 | 0.33 | 1.10 | 149.0 |
| TIZ | 15.0 | 0.151 | 0.813 | 100.0 | 28.0 | 0.19 | 0.92 | 142.0 | 27.0 | 0.23 | 1.00 | 119.0 |
| 3 | > 100.0 | 0.82 | 2.7 | > 124.0 | 11.0 | 0.60 | 2.60 | 18.3 | 9.9 | 0.42 | 3.30 | 23.5 |
| 4 | > 100.0 | 5.8 | 20.0 | > 17.0 | | | | | | | | |
| 6 | 11.0 | 0.31 | 1.2 | 35.0 | 0.64 | 0.10 | 0.61 | 6.5 | 0.79 | 0.11 | 0.82 | 6.9 |
| 7 | 34.0 | 1.10 | 6.1 | 31.0 | | | | | | | | |
| 9 | 40.0 | 1.90 | 9.0 | 21.0 | | | | | | | | |
| 14 | 35.0 | 2.80 | 7.8 | 13.0 | | | | | | | | |
| 15 | 78.0 | 2.90 | 11.0 | 27.0 | | | | | | | | |
| 20 | > 100.0 | 6.5 | 16.0 | > 15.0 | | | | | | | | |
| 23 | 72.0 | 3.8 | 21.0 | 19.0 | | | | | | | | |
| 24 | > 100.0 | 2.1 | 6.8 | > 49.0 | | | | | | | | |
| 25 | 5.8 | 0.13 | 0.82 | 43.0 | 3.4 | 0.078 | 0.31 | 43.6 | 4.3 | 0.10 | 0.46 | 43.0 |
| 27 | > 100.0 | 3.6 | 11.00 | > 28.0 | | | | | | | | |
| 34 | 4.3 | 0.44 | 1.70 | 9.7 | 9.5 | 0.91 | 3.70 | 10.5 | 7.8 | 0.79 | 4.7 | 9.9 |
| 41 | 0.33 | 0.005 | 0.032 | 65.2 | 0.79 | 0.045 | 0.177 | 17.5 | 0.68 | 0.055 | 0.244 | 12.3 |
| 44 | 4.4 | 0.058 | 0.461 | 75.9 | 6.8 | 0.35 | 4.5 | 19.4 | 7.5 | 0.30 | 5.1 | 24.8 |
| 49 | 3.9 | 0.313 | 1.10 | 12.0 | | | | | | | | |

The antiviral activity of salicylanilides against HBV in virion and replication intermediate cell culture assays is presented in Table 3.

TABLE 3

| | | EC50 | EC50 | EC90 | EC90 | | |
|---|---|---|---|---|---|---|---|
| | CC50 | (VIR) | (RI) | (VIR) | (RI) | SI | SI |
| No. | (uM) | (uM) | (uM) | (uM) | (uM) | (VIR) | (RI) |
| NTZ | > 100.0 | 0.12 | 0.59 | 0.83 | 2.10 | > 121.0 | > 48.0 |
| TIZ | > 100.0 | 0.15 | 0.46 | 0.58 | 1.20 | > 172.4 | > 83.3 |
| 38 | > 100.0 | 3.50 | | 9.7 | | > 11.0 | |
| 39 | > 100.0 | 0.76 | 2.1 | 3.0 | 6.8 | > 33.0 | > 15.0 |
| 40 | > 100.0 | 0.20 | 0.73 | 1.20 | 3.30 | > 83.0 | > 30.0 |
| 41 | > 100.0 | 3.7 | | 13.0 | | > 7.7 | |
| 42 | > 100.0 | 0.23 | 0.68 | 0.71 | 2.10 | > 141.0 | > 48.0 |
| 51 | > 100.0 | 5.6 | | 21.0 | | > 4.8 | |
| 52 | 59.0 | 2.5 | | 9.1 | | 65.0 | |
| 53 | 52.0 | 2.0 | | 8.7 | | 6.0 | |
| 54 | 65.0 | 2.1 | | 8.5 | | 7.6 | |
| 55 | 48.0 | 2.2 | | 8.8 | | 5.5 | |

1. A method for treating a viral infection comprising administering a compound of structural Formula (I): 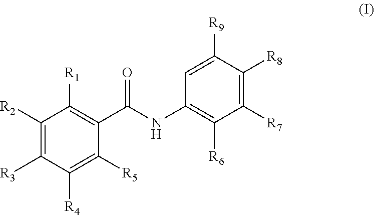
and pharmaceutically acceptable salts thereof,
wherein R₁, R₂ or R₃ are each independently selected from the group consisting of hydrogen, D, hydroxy, F, Cl, Br, (C₁-C₆)-alkyl, (C₃-C₈)-cycloalkyl, (C₁-C₆)-alkoxy, carboxy, (C₁-C₆)-alkoxycarbonyl, amino, (C₁-C₆)-acylamino, (C₁-C₆)-alkylsulfonylamino, and Q—C(═O)O—, any of which may be optionally substituted; or any two contiguous R₁, R₂ or R₃ moieties may be combined together with the atoms to which they are attached and joined to form an optionally substituted 5-to 8-membered heterocycloalkyl ring;
wherein R₄ and R₅ are independently selected from the group consisting of hydrogen, D, F, Cl, Br, (C₁-C₆)-alkyl, (C₃-C₈)-cycloalkyl, (C₁-C₆)-alkoxy, (C₂-C₆)-alkenyl, (C₂-C₆)-alkynyl and (C₁-C₁₂)-alkoxycarbonyl, any of which may be optionally substituted;
wherein R₆ through R₉ are independently selected from the group consisting of hydrogen, CN, NO₂, F, Cl, Br, I, (C₁-C₆)-alkyl, (C₂-C₆)-alkenyl, (C₂-C₆)-alkynyl, (C₃-C₈)-cycloalkyl, (C₃-C₈)-cycloalkenyl, (C₁-C₆)-alkoxy, (C₂-C₆)-alkenyloxy, (C₂-C₆)-alkynyloxy, (C₁-C₆)-alkoxyalkyl, (C₃-C₈)-cycloalkoxy, (C₅-C₈)-cycloalkenyloxy, (C₁-C₆)-alkoxyalkylamino, (C₁-C₆)-acyl, (C₁-C₆)-alkylamino, (C₁-C₆)-dialkylamino, carboxy, (C₁-C₆)-alkoxycarbonyl, amido, (C₁-C₆)-alkylamido, (C₁-C₆)-dialkylamido, (C₁-C₆)-haloalkyl, (C₁-C₆)-perhaloalkyl, (C₁-C₆)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₂-C₆)-alkenylsulfonyl, (C₂-C₆)-alkynylsulfonyl, (C₃-C₈)-cycloalkylsulfonyl, (C₃-C₈)-cycloalkylsulfinyl, arylsulfonyl, arylsulfinyl, arylalkylsulfonyl, arylalkylsulfinyl, arylalkenylsulfonyl, heteroarylsulfonyl, heteroarylsulfinyl, heteroarylalkylsulfonyl, heteroarylalkylsulfinyl, heteroarylalkenylsulfonyl, (C₁-C₆)-alkylsulfonamido, N,N′—(C₁-C₆)-dialkylsulfonamido, sulfonamidoalkyl, sulfonamidoaryl, sulfonamidoarylalkyl, sulfonamidoarylalkenyl, aryl, arylalkyl, aryloxy, arylalkoxy, arylthio, arylalkylthio, arylamino, arylalkylamino, arylalkenyl, arylalkynyl, heteroaryl, heteroarylalkyl, heteroarylalkenyl, heteroarylalkynyl, heteroaryloxy, heteroarylalkoxy, heteroarylamino, heteroarylalkylamino, heteroarylthio, heteroarylalkylthio, heteroarylalkylamino, heterocycloalkyl, heterocycloalkenyl, heterocycloalkoxy, and heterocycloalkenyloxy, any of which may be optionally substituted; and
wherein Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₁₂)-alkenyl, (C₂-C₁₂)-alkynyl, (C₃-C₈)-cycloalkyl, aryl, arylalkyl, arylalkenyl, heterocycloalkyl, heteroaryl, heteroarylalkyl, and heteroarylalkenyl, any of which may be optionally substituted; or R₁₀ and Ru, together with the atoms to which they are attached, may be joined to form an optionally substituted 5-to 8-membered heterocycloalkyl ring; any of which may be optionally substituted.

2. The method according to claim 1,
wherein R₁, R₂ or R₃ are each independently selected from the group consisting of hydrogen, D, hydroxy, F, Cl, Br, (C₁-C₃)-alkyl, (C₃-C₆)-cycloalkyl, (C₁-C₄)-alkoxy, carboxy, (C₁-C₃)-alkoxycarbonyl, (C₁-C₆)-acylamino, (C₁-C₆)-alkylsulfonylamino, and Q—C(═O)O—, any of which may be optionally substituted; or any two contiguous R₁, R₂ or R₃ moieties together with the atoms to which they are attached and joined to form an optionally substituted 5-to 6-membered heterocycloalkyl ring;
wherein R₄ and R₅ are independently selected from the group consisting of hydrogen, D, F, Cl, Br, (C₁-C₃)-alkyl, (C₃-C₆)-cycloalkyl, (C₁-C₃)-alkoxy, (C₂-C₄)-alkenyl, and (C₂-C₄)-alkynyl, any of which may be optionally substituted;
wherein R₆ through R₉ are independently selected from the group consisting of hydrogen, CN, NO₂, F, Cl, Br, I, (C₁-C₆)-alkyl, (C₃-C₆)-cycloalkyl, (C₁-C₆)-alkoxy, (C₃-C₆)-cycloalkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, arylsulfonyl, arylsulfinyl, arylalkylsulfonyl, arylalkylsulfinyl, heteroarylsulfonyl, heteroarylsulfinyl, heteroarylalkylsulfonyl, heteroarylalkylsulfinyl, (C₁-C₆)-alkylsulfonamido, N,N′—(C₁-C₆)-dialkylsulfonamido, aryl, arylalkyl, aryloxy, arylalkoxy, arylthio, arylalkylthio, heteroaryl, heteroarylalkyl, heteroaryloxy, heteroarylalkoxy, heteroarylthio, heteroarylalkylthio, heterocycloalkyl, and heterocycloalkoxy, any of which may be optionally substituted; and
wherein Q is R₁₀, OR₁₀, NHR₁₀, or NR₁₀R₁₁; R₁₀ and R₁₁ are independently selected from the group consisting of hydrogen, (C₁-C₁₂)-alkyl, (C₂-C₆)-alkenyl, (C₃-C₆)-cycloalkyl, aryl, arylalkyl, arylalkenyl, heterocycloalkyl, heteroaryl, heteroarylalkyl, and heteroarylalkenyl, any of which may be optionally substituted; or R₁₀ and R₁₁, together with the atoms to which they are attached, may be joined to form an optionally substituted 5- to 6-membered heterocycloalkyl ring; any of which may be optionally substituted.

3. The method according to claim 2,
wherein R₁ is selected from the group consisting of hydroxy, carboxy, (C₁-C₃)-alkoxycarbonyl, and Q—C(═O)O—, any of which may be optionally substituted;
wherein R₂ and R₃ are independently selected from the group consisting of hydrogen, hydroxy, D, F, Cl, Br, (C₁-C₃)-alkyl, (C₃-C₆)-cycloalkyl, (C₁-C₄)-alkoxy, and Q—C(═O)O—, any of which may be optionally substituted; or R₁ and R₂ may be combined together with the atoms to which they are attached and joined to form an optionally substituted 5-to 6-membered heterocycloalkyl ring; and
wherein R₄ and R₅ are independently selected from the group consisting of hydrogen, D, F, Cl, Br, (C₁-C₃)-alkyl, (C₃-C₆)-cycloalkyl, and (C₁-C₃)-alkoxy, any of which may be optionally substituted.

4. The method according to claim 3,
wherein R₁ is selected from the group consisting of hydroxy and Q—C(═O)O—, which may be optionally substituted;
wherein R₂ and R₃ are independently selected from the group consisting of hydrogen, D, F, Cl, (C₁-C₃)-alkyl, and (C₁-C₄)-alkoxy, any of which may be optionally substituted; and
wherein R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

5. The method according to claim 4,
wherein R₁ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

6. The method according to claim 5,
wherein R₂ and R₃ are independently selected from the group consisting of hydrogen, F, Cl, methyl, ethyl, isobutyl, methoxy, ethoxy, and isopropoxy, any of which may be optionally substituted;
wherein R₄ and R₅ are independently selected from the group consisting of hydrogen, F, Cl, Br, methyl, isopropyl, cyclopropyl and methoxy, any of which may be optionally substituted; and
wherein R₆ through R₉ are independently selected from the group consisting of hydrogen, CN, NO₂, F, Cl, Br, I, methyl, methoxy, methoxycarbonyl, butoxycarbonyl, amido, ethylamido, N,N-dimethylamido, trifluoromethyl, tetrafluoroethoxy, methylthio, ethylthiomethyl, methylsulfonyl, t-butylsulfinyl, 2-methylsulfonyl-1-ethyl, 2-methylsulfinylmethyl, cyclopropylsulfonyl, cyclopentylsulfinyl, N-propylsulfonamido, and N,N′-diethylsulfonamido, any of which may be optionally substituted.

7. The method according to claim 6,
wherein R₁ is selected from the group consisting of hydroxy and Q, where R₁₀ is methyl;
wherein R₂ and R₄ are independently selected from the group consisting of hydrogen, F, Cl, methyl, and methoxy;
wherein R₃ and R₅ are hydrogen;
wherein R₆ is selected from the group consisting of hydrogen, F, Cl, Br, methyl, and methylsulfonyl;
wherein R₇ is selected from the group consisting of hydrogen, F, Cl, Br, NO₂, methyl, trifluoromethyl, methylsulfonyl and methoxycarbonyl;
wherein R₈ is selected from the group consisting of hydrogen, F, NO₂, Cl, I, methylsulfonyl, methoxycarbonyl, tetrafluoroethoxy, and trifluoromethyl; and
wherein R₉ is chosen from the group consisting of hydrogen, Cl, methyl, trifluoromethyl, methoxycarbonyl, and NO₂.

8. The method according to claim 7, wherein R₂ through R₅ are hydrogen.

9. The method according to claim 2,
wherein R₂ is selected from the group consisting of hydroxy, carboxy, (C₁-C₃)-alkoxycarbonyl, and Q—C(═O)O—, any of which may be optionally substituted;
wherein R₁ and R₃ are independently selected from the group consisting of hydrogen, hydroxy, D, F, Cl, Br, (C₁-C₃)-alkyl, (C₃-C₆)-cycloalkyl, (C₁-C₄)-alkoxy, and Q—C(═O)O—, any of which may be optionally substituted; or R₂ and R₃ may be combined together with the atoms to which they are attached and joined to form an optionally substituted 5-to 6-membered heterocycloalkyl ring; and
wherein R₄ and R₅ are independently selected from the group consisting of hydrogen, D, F, Cl, Br, (C₁-C₃)-alkyl, (C₃-C₆)-cycloalkyl, and (C₁-C₃)-alkoxy, any of which may be optionally substituted.

10. The method according to claim 9,
wherein R₂ is selected from the group consisting of hydroxy and Q—C(═O)O—, which may be optionally substituted;
wherein R₁ and R₃ are independently selected from the group consisting of hydrogen, D, F, Cl, (C₁-C₃)-alkyl, and (C₁-C₄)-alkoxy, any of which may be optionally substituted; and
wherein R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

11. The method according to claim 10,
wherein R₂ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

12. The method according to claim 11,
wherein R₁ and R₃ are independently selected from the group consisting of hydrogen, F, Cl, methyl, ethyl, isobutyl, methoxy, ethoxy, and isopropoxy, any of which may be optionally substituted;
wherein R₄ and R₅ are independently selected from the group consisting of hydrogen, F, Cl, Br, methyl, isopropyl, cyclopropyl and methoxy, any of which may be optionally substituted; and
wherein R₆ through R₉ are independently selected from the group consisting of hydrogen, CN, NO₂, F, Cl, Br, I, methyl, methoxy, methoxycarbonyl, butoxycarbonyl, amido, ethylamido, N,N-dimethylamido, trifluoromethyl, tetrafluoroethoxy, methylthio, ethylthiomethyl, methylsulfonyl, t-butylsulfinyl, 2-methylsulfonyl-1-ethyl, 2-methylsulfinylmethyl, cyclopropylsulfonyl, cyclopentylsulfinyl, N-propylsulfonamido, and N,N′-diethylsulfonamido, any of which may be optionally substituted.

13. The method according to claim 12,
wherein R₂ is selected from the group consisting of hydroxy and Q, where R₁₀ is methyl;
wherein R₁, R₃, and R₄ are independently selected from the group consisting of hydrogen, F, Cl, methyl, methoxy and ethoxy, any of which may be optionally substituted;
wherein R₅ is hydrogen;
wherein R₆ is selected from the group consisting of hydrogen, F, Cl, Br, methyl, and methylsulfonyl;
wherein R₇ is selected from the group consisting of hydrogen, F, Cl, Br, NO₂, methyl, trifluoromethyl, methylsulfonyl and methoxycarbonyl;
wherein R₈ is selected from the group consisting of hydrogen, F, NO₂, Cl, I, methylsulfonyl, methoxycarbonyl, tetrafluoroethoxy, and trifluoromethyl; and
wherein R₉ is selected from the group consisting of hydrogen, Cl, methyl, trifluoromethyl, methoxycarbonyl, and NO₂.

14. The method according to claim 13, wherein R₁ and R₃ through R₅ are hydrogen.

15. The method according to claim 2,
wherein R₃ is selected from the group consisting of hydroxy, carboxy, (C₁-C₃)-alkoxycarbonyl, and Q—C(═O)O—, any of which may be optionally substituted;
wherein R₁ and R₂ are independently selected from the group consisting of hydrogen, hydroxy, D, F, Cl, Br, (C₁-C₃)-alkyl, (C₃-C₆)-cycloalkyl, (C₁-C₄)-alkoxy, and Q—C(═O)O—, any of which may be optionally substituted; or R₁ and R₂ may be combined together with the atoms to which they are attached and joined to form an optionally substituted 5-to 6-membered heterocycloalkyl ring; and
wherein R₄ and R₅ are independently selected from the group consisting of hydrogen, D, F, Cl, Br, (C₁-C₃)-alkyl, (C₃-C₆)-cycloalkyl, and (C₁-C₃)-alkoxy, any of which may be optionally substituted.

16. The method according to claim 15,
wherein R₃ is selected from the group consisting of hydroxy and Q—C(═O)O—, which may be optionally substituted;
wherein R₁ and R₂ are independently selected from the group consisting of hydrogen, D, F, Cl, (C₁-C₃)-alkyl, and (C₁-C₄)-alkoxy, any of which may be optionally substituted; and
wherein R₆ through R₉ are independently selected from the group consisting of hydrogen, CN, NO₂, F, Cl, Br, I, (C₁-C₆)-alkyl, (C₁-C₆)-alkoxy, carboxy, (C₁-C₆)-alkoxycarbonyl, amido, (C₁-C₆)-alkylamido, (C₁-C₆)-dialkylamido, (C₁-C₃)-haloalkyl, (C₁-C₃)-perhaloalkyl, (C₁-C₄)-perhaloalkoxy, (C₁-C₆)-alkylthio, (C₁-C₆)-alkylthioalkyl, (C₁-C₆)-alkylsulfonyl, (C₁-C₆)-alkylsulfinyl, (C₁-C₆)-alkylsulfonylalkyl, (C₁-C₆)-alkylsulfinylalkyl, (C₃-C₆)-cycloalkylsulfonyl, (C₃-C₆)-cycloalkylsulfinyl, (C₁-C₆)-alkylsulfonamido, and N,N′-(C₁-C₆)-dialkylsulfonamido, any of which may be optionally substituted.

17. The method according to claim 16,
wherein R₃ is selected from the group consisting of hydroxy, and Q is selected from hydrogen, methyl, ethyl, n-propyl, isopropyl, n-butyl, 2-methyl-1-propyl, sec-butyl, tert-butyl, 2,3-dimethylbutan-2-yl, cyclohexyl, 2,6-dimethylcyclohexyl, 1-methylcyclohexyl, phenyl, 4-pyridyl, benzyl, 4-pyridylmethyl, phenylethyl, (S)-1-hydroxy-(phenylethyl), 2-pyrazinyl, phenylethenyl, (E)-2-(4-pyridazinyl)-1-ethenyl, (E)-4-(2-)-1H-imidazolyl-1-ethenyl, 3-acetoxyl-1-propyl, ethoxycarbonylethyl, methoxylcarbonylpropyl, N-methylaminocarbonylethyl, N-ethylaminocarbonylpropyl, 3-(N-ethylaminocarbonyl)-2,2-dimethyl-1-propyl, N-(morpholinoethyl)aminocarbonylethyl, 3-pyridylmethylaminocarbonylethyl, 4-pyridylmethylaminocarbonylethyl, 4-pyridylmethylaminocarbonylpropyl, carboxyethyl, carboxypropyl, 2-piperidinyl, 3-piperidinyl, 4-piperidinyl, 2-piperazinyl, (S)-1-aminoethyl, (R)-1-aminoethyl, (S)-1-aminoisobutyl, 1-aminocyclopropyl, methoxy, ethoxy, isopropoxy, isobutoxy, neopentyloxy, cyclohexyloxy, 4-piperidinyloxy, 3-acetoxy-2-methyl-1-propoxy, tert-pentyloxy, 4-acetoxybenzyloxy, 3-(4-acetoxyphenyl)-2-propenyloxy, (E)-2-methyl-4-(2-oxo-2,3-dihydrobenzofuran-5-yl)but-3-en-2-yloxy, pivaloyloxymethoxy, pivaloyloxy-1-ethoxy, isopropoxycarbonyloxymethoxy, isopropoxycarbonyloxy-1-ethoxy, amino, N-methylamino, N-ethylamino, N,N-dimethylamino, N-propylamino, N-isopropylamino, N-butylamino, N-piperidinyl, N-piperazinyl, N-4-methylpiperazinyl, N-cyclohexylamino, N-benzylamino, N-(2,4-dimethoxy)benzylamino, 2-(N-methylcarboxamido)phenylamino, N-methyl-2-hydroxyethylamino, N¹,N²-dimethyl-1,2-ethanediamin-1-yl, N¹,N²-diethyl-1,2-ethanediamin-1-yl, N¹,N²-dimethyl-1,3-propanediamin-1-yl, and N¹-methyl-N²-(2-morpholinoethyl)-1,2-ethanediamin-1-yl.

18. The method according to claim 17,
wherein R₁ and R₂ are independently selected from the group consisting of hydrogen, F, Cl, methyl, ethyl, isobutyl, methoxy, ethoxy, and isopropoxy, any of which may be optionally substituted;
wherein R₄ through R₅ are independently selected from the group consisting of hydrogen, F, Cl, Br, methyl, isopropyl, cyclopropyl and methoxy, any of which may be optionally substituted; and
wherein R₆ through R₉ are independently selected from the group consisting of hydrogen, CN, NO₂, F, Cl, Br, I, methyl, methoxy, methoxycarbonyl, butoxycarbonyl, amido, ethylamido, N,N-dimethylamido, trifluoromethyl, tetrafluoroethoxy, methylthio, ethylthiomethyl, methylsulfonyl, t-butylsulfinyl, 2-methylsulfonyl-1-ethyl, 2-methylsulfinylmethyl, cyclopropylsulfonyl, cyclopentylsulfinyl, N-propylsulfonamido, and N,N′-diethylsulfonamido, any of which may be optionally substituted.

19. The method according to claim 18,
wherein R₃ is selected from the group consisting of hydroxy and Q, where R₁₀ is methyl;
wherein R₁, R₂, and R₄ are independently selected from the group consisting of hydrogen, F, Cl, methyl, methoxy and ethoxy, any of which may be optionally substituted;
wherein R₅ is hydrogen;
wherein R₆ is selected from the group consisting of hydrogen, F, Cl, Br, methyl, and methylsulfonyl;
wherein R₇ is selected from the group consisting of hydrogen, F, Cl, Br, NO₂, methyl, trifluoromethyl, methylsulfonyl and methoxycarbonyl;
wherein R₈ is selected from the group consisting of hydrogen, F, NO₂, Cl, I, methylsulfonyl, methoxycarbonyl, tetrafluoroethoxy, and trifluoromethyl; and
wherein R₉ is selected from the group consisting of hydrogen, Cl, methyl, trifluoromethyl, methoxycarbonyl, and NO₂.

20. The method according to claim 19, wherein R₁, R₂, R₄ and R₅ are hydrogen.

21. The method according to claim 1, wherein the compound is selected from Examples 1 through 55 in Table 1.

22. The method according to claim 1, where the compound is selected from Examples 56 through 123 in Table 4.

23. The method according to claim 1,
wherein the salt is selected from the group consisting of acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethane-sulfonate, lactate, malate, maleate, methanesulfonate, 2-naphthalenesulfonate, nicotinate, oxalate, thiocyanate, tosylate, undecanoate, lithium, sodium, calcium, potassium, aluminum, ammonium, tetraethylammonium, methylammonium, dimethylammonium, N-methylmorpholinium and ethanolammonium.

24. A pharmaceutical composition comprising a therapeutically effective amount of at least one compound according to claim 1, and a pharmaceutically acceptable carrier.

25. The pharmaceutical composition of claim 24, for treating an infection in a subject exposed to a viral pathogen.

26. The pharmaceutical composition of claim 25, wherein the viral pathogen is Hepatitis C Virus infection.

27. The pharmaceutical composition of claim 25, wherein the viral pathogen is Hepatitis B Virus infection.

28. A method for treating a viral infection comprising administering the pharmaceutical composition of claim 25 to a patient in need thereof.

29. The method as recited in claim 28 wherein the viral infection is Hepatitis C Virus.

30. The method as recited in claim 28 wherein the viral infection is Hepatitis B Virus.
